(19) United States
(12) Patent Application Publication
(10) Pub. No.: US 2022/0152077 A1
(43) Pub. Date: May 19, 2022

(54) ADENOSINE RECEPTOR AGONISTS

(71) Applicant: THE UNIVERSITY OF WARWICK, Coventry, West Midlands (GB)

(72) Inventors: Bruno FRENGUELLI, Coventry (GB); Mark WALL, Coventry (GB)

(21) Appl. No.: 17/441,257

(22) PCT Filed: Mar. 19, 2020
(86) PCT No.: PCT/GB2020/050734

§ 371 Date: Sep. 20, 2021

(30) Foreign Application Priority Data
Mar. 21, 2019 (GB) 1903900.7

(51) Int. Cl. A61K 31/7076; A61P 25/02; A61P 25/08; A61P 25/16; A61P 25/18; A61P 25/20; A61P 25/28; A61P 29/02

(52) U.S. Cl. CPC A61K 31/7076; A61P 25/02; A61P 25/08; A61P 29/02; A61P 25/18; A61P 25/20; A61P 25/28; A61P 25/16

(57) ABSTRACT

The present invention relates to compounds of Formula (I) for use in the treatment of nervous system disorders and pain, wherein Formula (I) is:

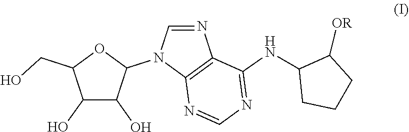

or a pharmaceutically acceptable salt or isomer thereof, wherein R is defined herein. The compounds are selective A₁ adenosine receptor agonists with preferential action in the nervous system, with spared cardiovascular system and respiratory effects. The invention also relates to pharmaceutical compositions comprising the compounds.

This invention relates generally to adenosine receptor agonists, methods for their manufacture, their uses for example as medicaments, and uses in the treatment of nervous system disorders and pain.

The purine nucleoside adenosine is a potent neuromodulator involved in many physiological processes and nervous system pathologies including pain, epilepsy and stroke (cerebral ischemia) (see, for example: J. Sawynok, Neuroscience, 2016, 338, 1-18; D. Boison, Neuropharmacology, 2016, 104, 131-139; Borea et al., Trends Pharmacol. Sci., 2016, 37, 419-434; N. Dale et al., Curr. Neuropharmacol., 2009, 7, 160-179). Adenosine acts via multiple subtypes of cell surface G protein coupled receptors (GPCRs) termed A₁, A_{2A}, A_{2B} and A₃, with the A₁ receptor (A₁R) having the widest distribution in the brain (see, for example: B. B. Fredholm et al., N-S Arch. Pharmacol., 2000, 362, 364-374).

During epileptic seizure activity, adenosine is released to activate A₁Rs on neurons which acts as a negative feedback mechanism to terminate the current burst of activity and delay the occurrence of the next burst of activity (see, for example: M. J. During et al., Ann. Neurol., 1992, 32, 618-624; N. Dale et al., 2009; D. Boison, 2016; M. J. Wall et al., J. Neurophysiol., 2015, 113, 871-882). In the hippocampus (which is commonly affected in epilepsy), activation of presynaptic A₁Rs depresses glutamatergic synaptic transmission to pyramidal cells, whilst activation of postsynaptic A₁Rs hyperpolarises the membrane potential of pyramidal cells through the activation of specific K⁺ channels (see, for example: G. R. Siggins et al., Neurosci. Lett., 1981, 23, 55-60; W. R. Proctor et al., Brain Res., 1987, 426, 187-190; T. V. Dunwiddie et al., J. Pharmac. Exp. Ther., 1989, 249, 31-37; S. M. Thompson et al., J. Physiol., 1992, 451, 347-363). The relative contribution of these two processes to the suppression of seizures remains unclear, as it is currently not possible to pharmacologically dissect apart these two effects of A₁R activation. Pre- and postsynaptic A₁ receptors may produce their effects through different second messengers and G proteins although this currently remains unclear.

A small number of N⁶-bicyclic and N⁶-(2-hydroxy)-cyclopentyl derivatives of adenosine have been reported to have a high potency and selectivity for recombinant A₁Rs (see, for example: A. Knight et al., J. Med. Chem., 2016, 59, 947-964). It would be advantageous to determine if such ligands showed signalling bias at A₁Rs (i.e. preferential activation of pre- or postsynaptic receptors in, for example, the brain). As these would be valuable tools for establishing the importance of the pre- and postsynaptic effects of adenosine at native receptors in intact tissue and for their therapeutic potential in nervous system disorders and pain.

As well as being expressed at a high density in the nervous system, adenosine A₁ receptors also have high expression in the cardiovascular system (CVS), particularly in cardiac tissue where they act to slow heart rate (bradycardia). The activation of the widely-distributed A₁ receptor (A₁R) with currently available agonists therefore elicits multiple actions in both the nervous system, such as inhibition of synaptic transmission and neuronal hyperpolarization, and the cardiorespiratory system through slowing the heart (bradycardia), reducing blood pressure (hypotension) and affecting respiration (dyspnea). These multiple effects severely limit the prospects of A₁R agonists as life-changing medicines, despite their potential in a wide range of clinical conditions including pain, epilepsy and cerebral ischemia. Therefore, it would be advantageous to provide ligands with nervous system selectivity and with spared CVS and respiratory side-effects.

The therapeutic limitations of promiscuous G protein-coupled receptor coupling may be overcome through the development of biased agonists. Biased agonists are compounds that selectively recruit one intracellular signalling cascade over another. However, to date no A₁R-specific agonist has been reported that can elicit Ga biased agonism in intact physiological systems.

An object of the present invention is to provide an adenosine A₁ receptor agonist that displays signalling bias within an apparent preferential action in the nervous system with spared CVS and/or respiratory effects, which can be used in the treatment of nervous system disorders and pain.

WO2011/119919 describes benzyloxy cyclopentyladenosine (BCPA) compounds and their use as selective A₁ adenosine receptor agonists.

Accordingly, a first aspect of the invention provides a compound e.g. an adenosine receptor agonist, represented by the following general Formula (I), for use in the treatment of a nervous system disorder or pain, wherein Formula (I) is:

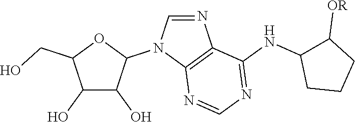

(I), or a pharmaceutical acceptable salt or isomer thereof, wherein:

R is independently hydrogen or R¹R²R³, wherein:
i. R¹ is independently C₁₋₁₀ alkyl;
ii. R² is independently aryl; and
iii. R³ is independently hydrogen, OH, C(O)NH₂, linear or branched C₁-C₁₀ alkyl, or C₃-C₈ cycloalkyl.

In some embodiments, R is hydrogen.

In some embodiments, R is R¹R²R³.

In some embodiments, R¹ in the above identified general formula represents an alkyl group. The alkyl group having 1 to 10 carbon atoms represented by R¹ may be a linear or branched alkyl group having 1 to 5 carbon atoms. Specific examples of the alkyl group having 1 to 10 carbon atoms include a methyl group, an ethyl group, a n-propyl group, an isopropyl group, a n-butyl group, an isobutyl group, a s-butyl group, a t-butyl group, a n-pentyl group, a n-hexyl group, a n-heptyl group, a n-octyl group, a n-nonyl group, a n-decyl group, and the like. The alkyl group may be a methyl group, an ethyl group, a n-propyl group, an isopropyl group, a n-butyl group, an isobutyl group, a s-butyl group, a t-butyl group, or a n-pentyl group.

In some embodiments, R¹ is CH₂.

In some embodiments, R² in the above identified general formula represents an aryl group. The aryl group having 6 to 30 carbon atoms may be an aromatic monocyclic group, aromatic polycyclic group, or aromatic fused cyclic group having 6 to 30 carbon atoms, and is preferably an aromatic monocyclic group, aromatic polycyclic group, or aromatic fused cyclic group having 6 to 15 carbon atoms, for example an aromatic monocyclic group having 6 to 12 carbon atoms. Specific examples of the aryl group having 6 to 30 carbon atoms include a phenyl group, a naphthyl group, an anthryl group, a phenanthryl group, an indenyl group, and the like.

In some embodiments, R² is a phenyl group.

In some embodiments, R³ in the above identified general formula may represent an alkyl group. The alkyl group having 1 to 10 carbon atoms represented by R³ is preferably a linear or branched alkyl group having 1 to 5 carbon atoms. Specific examples of the alkyl group having 1 to 10 carbon atoms include a methyl group, an ethyl group, a n-propyl group, an isopropyl group, a n-butyl group, an isobutyl group, a s-butyl group, a t-butyl group, a n-pentyl group, a n-hexyl group, a n-heptyl group, a n-octyl group, a n-nonyl group, a n-decyl group, and the like. The alkyl group may be a methyl group, an ethyl group, a n-propyl group, an isopropyl group, a n-butyl group, an isobutyl group, a s-butyl group, a t-butyl group, or a n-pentyl group.

In some embodiments, R³ is a branched C₁-C₁₀ alkyl.

In some embodiments, R³ is a branched C₃-C₄ alkyl.

In some embodiments, R³ in the above identified general formula may represent a cycloalkyl group. The cycloalkyl group having 3 to 8 carbon atoms represented by R³ may be a monocyclic, polycyclic, or bridged cycloalkyl group having 5 to 8 carbon atoms.

In some embodiments, the cycloalkyl group is a monocyclic cycloalkyl group having 3 to 8 carbon atoms. Specific examples of the cycloalkyl group having 3 to 8 carbon atoms include a cyclopropyl group, a cyclobutyl group, a cyclopentyl group, a cyclohexyl group, a cycloheptyl group, a cyclooctyl group, and the like.

In some embodiments, R³ is C₃-C₈ cycloalkyl.

In some embodiments, R³ is cyclopropyl.

In some embodiments, R³ is OH.

In some embodiments, R³ is C(O)NH₂.

In some embodiments, R³ is t-butyl.

Exemplar compounds within the scope of Formula (I) include:

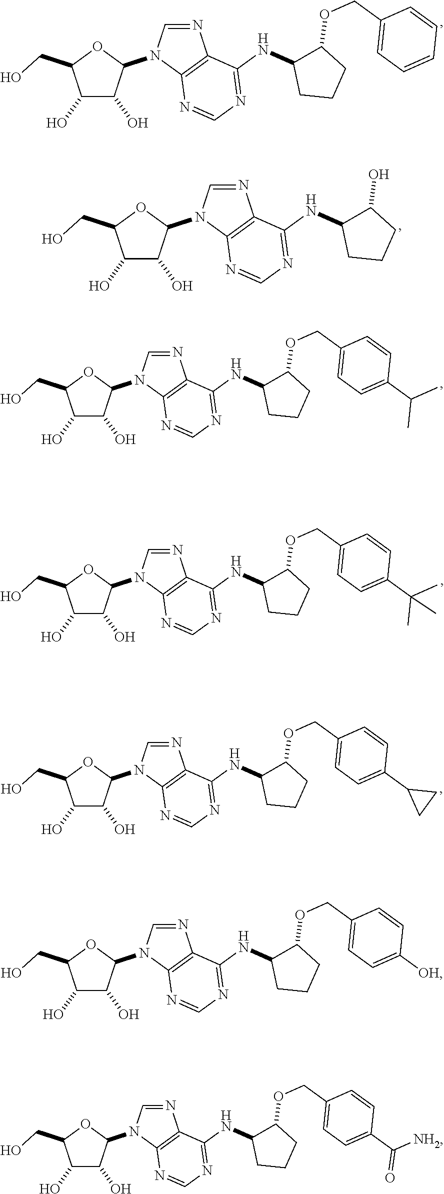

or pharmaceutically acceptable salts thereof.

In embodiments, the compound of Formula (I) is not the following compound:

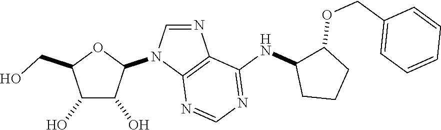

According to a further aspect of the invention, there is provided a pharmaceutical composition comprising a compound of Formula (I) as described herein, and a pharmaceutically or therapeutically acceptable excipient or carrier.

The compounds of the invention are adenosine receptor agonists. In some embodiments, the compound of Formula (I) is an agonist of the A1 receptor (A₁R). Like known A₁R agonists, the compound of Formula (I) may inhibit synaptic transmission.

In some embodiments, the compound of Formula (I) is for use in the treatment of a nervous system disorder.

The nervous system disorder may be selected from the group consisting of epilepsy, ischemia (e.g. stroke), traumatic brain injury (TBI), hypoxia, Parkinson's disease, Alzheimer's disease, Huntington's disease, multiple sclerosis, cerebral palsy (and other disorders that may cause spasticity, such as encephalitis, meningitis, adrenoleukodystrophy, amyotrophic lateral sclerosis, phenylketonuria), spinal cord injury, dementia, schizophrenia and sleep disorders including insomnia.

In some embodiments, the nervous system disorder is epilepsy, ischemia or TBI.

The invention also encompasses a method of treating a nervous system disorder or pain, comprising the step of administering a therapeutically effective amount of the compound or the pharmaceutical composition as defined herein to a patient in need of same.

It has been surprisingly found by the present inventors that a compound of Formula (I), BnOCPA, inhibits synaptic transmission via activation of presynaptic A₁Rs, but does not activate postsynaptic A₁Rs to induce membrane hyperpolarization. Instead, this compound was observed to function in a manner analogous to a receptor antagonist at postsynaptic A₁Rs. The inventors further surprisingly demonstrated that the compound is highly selective, being able to activate the Ga subunit Gob but not Goa, unlike known A₁R agonists (Table 1). Without being bound by theory, it is believed that this selectivity for Gob explains why the compound does not cause membrane potential hyperpolarisation, which is thought to occur mainly through A₁R activation of the subunit Goa. BnOCPA thus exhibits strong bias between individual Ga subunits. It will be appreciated by those skilled in the art that the Ga coupling of A₁Rs is more important than their localisation, and that the action of A₁Rs on cells, tissues and organs is dependent upon the Ga subunits present within those structures and associated with A₁Rs, and not the location of the A₁Rs within those structures.

The subunit selectivity of BnOCPA is particularly significant, since a key obstacle to the use of A₁R agonists in treating conditions such as nervous system disorders and pain is the side effects in the CVS, for example reduced heart rate and blood pressure, and altered respiration. These side effects are likely effected through Goa which is expressed at high levels in the heart. However, unlike known A₁R agonists, BnOCPA was found not to activate A₁Rs in the heart, and had no effect on heart rate, blood pressure or respiration.

It has therefore been surprisingly found that compounds according to the invention are useful in the treatment of nervous system disorders and pain, without causing unwanted side effects in the CVS or respiratory system. These observations make the compounds of the invention particularly suited for use in the treatment of patients who, in addition to suffering from a nervous system disorder or pain, are suffering from or are at risk of a cardiovascular or respiratory disease.

Thus, in some embodiments, the compound of Formula (I) does not activate Goa. For example, the compound may activate Gob but not Goa. The skilled person is able to determine the ability of a compound to activate a given G protein subunit using their common general knowledge, or the methods described herein.

In some embodiments, the compound of Formula (I) does not activate any of: Gi1, Gi2, Gi3, Goa or Gz.

In some embodiments, the compound of Formula (I) only activates Gob.

As is known by a person skilled in the art, the terms “Goa” and “Gob” refer to different isoforms of the G protein subunit Ga. The skilled person will further appreciate that the compounds of the invention, and other A₁R agonists, do not activate G protein subunits directly, but exert their effects indirectly by binding to the A₁ receptor. The A₁R-agonist complex then activates the G protein subunits.

The compound of Formula (I) may be capable of inhibiting synaptic transmission. The ability of a compound to inhibit synaptic transmission can be determined by the skilled person using the methods described herein.

In some embodiments, the compound of Formula (I) is capable of activating presynaptic A₁R but not post-synaptic A₁Rs. The compound of Formula (I) may be, or may function in a manner analogous to, an antagonist of post-synaptic A₁Rs. The activation of A₁Rs can be determined using the methods described herein,

In some embodiments, the compound of Formula (I) is not capable of inducing membrane hyperpolarisation. The ability of a compound to induce membrane hyperpolarisation may be assessed using the methods described herein.

In some embodiments, the compound is not:

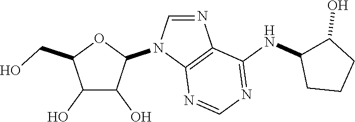

In some embodiments, the invention provides a compound represented by the following general Formula (I) for use in the treatment of a nervous system disorder or pain, wherein said use does not cause at least one of the following side effects: bradycardia, hypotension and dyspnea.

Further provided is a compound as defined herein for use in the treatment of a nervous system disorder or pain, wherein the patient to be treated is also suffering from, is at risk of, or is in need of treatment for, a cardiovascular or respiratory disease.

The cardiovascular disease may be selected from the group consisting of acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder and transitory ischemic attacks.

The term “respiratory disease” shall be interpreted to mean any pulmonary disease or impairment of lung function. Such diseases can be broadly divided into restrictive and obstructive disease. The respiratory disease may be an obstructive disease such as chronic obstructive pulmonary disease (COPD), pulmonary emphysema, chronic bronchitis, bronchiectasis, bronchiolitis, cystic fibrosis, or asthma. The respiratory disease may be a restrictive disease such as interstitial lung disease (e.g. idiopathic pulmonary fibrosis), sarcoidosis, scoliosis, neuromuscular disease (e.g. muscular dystrophy or amylotrophic lateral sclerosis (ALS)), or tuberculosis.

In some embodiments, the compound of Formula (I) is for use in the treatment of pain.

The pain may be selected from the group consisting of neuropathic, nociceptive, peripheral acute, chronic, somatic, visceral, neuroma, diabetic neuropathy, surgical pain, chemotherapy-induced pain, bone pain (e.g. fracture or cancer), inflammatory, phantom limb, myalgia, and multiple sclerosis-related pain.

In some embodiments, the pain is nociceptive pain or neuropathic pain, e.g. chronic neuropathic pain.

Therapeutically effective amounts may be determined by one skilled in the art by, for example, starting with the dosage range described in this specification for the compound of Formula (I) or pharmaceutically acceptable salt thereof.

Compounds of the invention may be administered at a dose of from 2 to 1000 μg/kg, from 3 to 500 μg/kg, from 4 to 100 μg/kg or from 5 to 50 μg/kg. In some embodiments, compounds of the invention are administered at a dose of from 5 to 20 ag/kg or from 8 to 15 μg/kg, e.g. 10 μg/kg. One, two, three or more doses may be administered per day. Doses may be administered on two or more consecutive days, for a period sufficient to deliver treatment, as determined by a person skilled in the art.

A third aspect of the invention provides a compound of Formula (I) as described herein.

A fourth aspect of the invention provides a compound of Formula (I) as described herein for use as a medicament.

In some embodiments of the third or fourth aspect of the invention, R is R¹R²R³, wherein:
i. R¹ is C₁₋₁₀ alkyl;
ii. R² is aryl; and
iii. R³ is independently hydrogen, OH, C(O)NH₂, linear or branched C₁-C₁₀ alkyl, or C₃-C₈ cycloalkyl.

In some embodiments of the third or fourth aspect of the invention, R is R¹R²R³, wherein:
i. R¹ is CH₂;
ii. R² is phenyl; and
iii. R³ is independently hydrogen, OH, C(O)NH₂, linear or branched C₁-C₁₀ alkyl (e.g. branched C₄-C₁₀ alkyl, such as t-butyl), or C₃-C₈ cycloalkyl (such as cyclopropyl).

In some embodiments of the third or fourth aspect of the invention, the compound of Formula (I) is selected from the group consisting of:

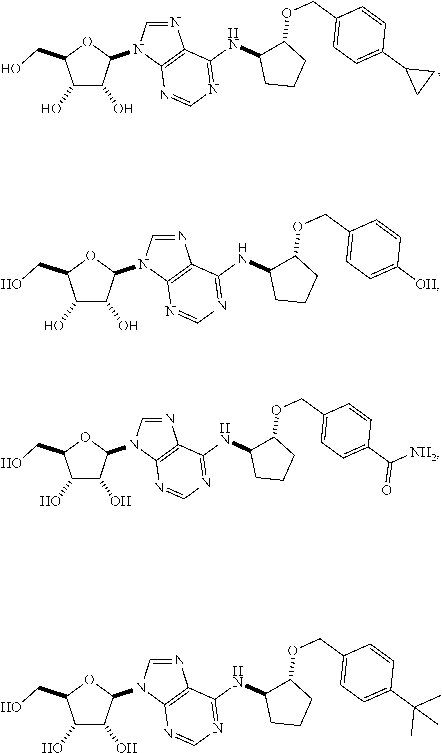

or pharmaceutically acceptable salts or isomers thereof.

In some embodiments of the third or fourth aspect of the invention, the compound is not:

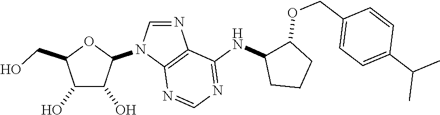

Further provided is the use of a compound of Formula (I), as defined herein, in the manufacture of a medicament for the treatment of a disease or condition.

The invention also encompasses a method of treating a disease or condition, comprising the step of administering a therapeutically effective amount of the compound or the pharmaceutical composition as defined herein to a patient in need of same.

Diseases and conditions suitable for treatment according to the relevant aspects of the invention are nervous system disorders and pain.

The following definitions shall apply throughout the specification and the appended claims.

As used herein, the term “comprising” is to be read as meaning both comprising and consisting of. Consequently, where the invention relates to a “composition comprising a compound”, this terminology is intended to cover both compositions in which other active ingredients may be present and also compositions which consist only of one active ingredient as defined. Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference (where legally permissible).

Unless otherwise stated or indicated, the term “alkyl” means a monovalent saturated, linear or branched, carbon chain, such as C₁₋₈, C₁₋₆ or C₁₋₄, which may be unsubstituted or substituted. The group may be partially or completely substituted with substituents independently selected from one or more of halogen (F, Cl, Br or I), hydroxy, nitro and amino. Non-limiting examples of alkyl groups methyl, ethyl, n-propyl, iso-propyl, n-butyl, iso-butyl, sec-butyl, tert-butyl, n-pentyl, iso-pentyl, n-pentyl, n-hexyl, etc. An alkyl group preferably contains from 1 to 6 carbon atoms, e.g. 1 to 4 carbon atoms.

Unless otherwise stated or indicated, the term “cycloalkyl” refers to a monovalent, saturated cyclic carbon system. Unless otherwise specified, any cycloalkyl group may be substituted in one or more positions with a suitable substituent. Where more than one substituent group is present, these may be the same or different. Suitable substituents include halogen (F, Cl, Br or I), hydroxy, nitro and amino.

Unless otherwise stated or indicated, the term “aryl” is intended to cover aromatic ring systems. Such ring systems may be monocyclic or polycyclic (e.g. bicyclic) and contain at least one unsaturated aromatic ring. Where these contain polycyclic rings, these may be fused. Preferably such systems contain from 6 to 20 carbon atoms, e.g. either 6 or 10 carbon atoms. Examples of such groups include phenyl, 1-naphthyl, 2-naphthyl and indenyl. A preferred aryl group is phenyl.

The term “therapeutically effective amount” means an amount of an agent or compound which provides a therapeutic benefit in the treatment of a disease, wherein the disease is selected from the group consisting of nervous system disorders or pain.

The term “pharmaceutically acceptable” means being useful in preparing a compound or pharmaceutical composition that is generally safe, non-toxic and neither biologically nor otherwise undesirable and includes being useful for veterinary use as well as human pharmaceutical use.

The term “pharmaceutically acceptable salt” comprises, but is not limited to, soluble or dispersible forms of compounds according to Formula (I) that are suitable for treatment of disease without undue undesirable effects such as allergic reactions or toxicity. Representative pharmaceutically acceptable salts include, but are not limited to, acid addition salts such as acetate, citrate, benzoate, lactate, or phosphate and basic addition salts such as lithium, sodium, potassium, or aluminium.

The term “pharmaceutically or therapeutically acceptable excipient or carrier” refers to a solid or liquid filler, diluent or encapsulating substance which does not interfere with the effectiveness or the biological activity of the active ingredients and which is not toxic to the host, which may be either humans or animals, to which it is administered. Depending upon the particular route of administration, a variety of pharmaceutically-acceptable carriers such as those well known in the art may be used. Non-limiting examples include sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

All suitable modes of administration are contemplated according to the invention. For example, administration of the medicament may be via oral, subcutaneous, direct intravenous, slow intravenous infusion, continuous intravenous infusion, intravenous or epidural patient controlled analgesia (PCA and PCEA), intramuscular, intrathecal, epidural, intracistemal, intraperitoneal, transdermal, topical, transmucosal, buccal, sublingual, inhalation, intra-atricular, intranasal, rectal or ocular routes. The medicament may be formulated in discrete dosage units and can be prepared by any of the methods well known in the art of pharmacy. All suitable pharmaceutical dosage forms are contemplated. Administration of the medicament may for example be in the form of oral solutions and suspensions, tablets, capsules, lozenges, effervescent tablets, transmucosal films, suppositories, buccal products, oral mucoretentive products, topical creams, ointments, gels, films and patches, transdermal patches, abuse deterrent and abuse resistant formulations, sterile solutions suspensions and depots for parenteral use, and the like, administered as immediate release, sustained release, delayed release, controlled release, extended release and the like.

The term “isomer” used herein refers to all forms of structural and spatial isomers. In particular, the term “isomer” is intended to encompass stereoisomers. With regards to stereoisomers, a number of the compounds herein described may have one or more asymmetric carbon atoms and may occur as racemates, racemic mixtures and as individual enantiomers or diastereomers. All such isomeric forms are included within the present invention, including mixtures thereof. Furthermore, diastereomers and enantiomer products can be separated by chromatography, fractional crystallisation or other methods known to those of skill in the art.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimise potential damage to uninfected cells and, thereby, reduce side effects.

The pharmaceutical compositions can be included in a container, pack, or dispenser together with instructions for administration. According to this aspect, the invention provides a kit comprising at least one compound according to the invention or a pharmaceutical composition of the invention, optionally in addition to one or more further active agents as defined herein, preferably with instructions for the administration thereof in the therapeutic treatment of the human or animal body, e.g. the treatment of nervous system disorders and/or pain, as hereinbefore defined.

The term “treatment” or “treating” means any treatment of a disease in a subject, including:
(i) Preventing the disease, that is, causing the clinical symptoms of the disease not to develop;
(ii) Inhibiting the disease, that is, arresting the development of clinical symptoms; and/or
(iii) Relieving the disease, that is, causing the regression of clinical symptoms.

As used herein, the term “therapeutically effective amount” refers to an amount of a compound or composition as described in any of the embodiments herein which is effective to provide therapy in a subject. In the case of a nervous system disorder, the therapeutically effective amount may cause any of the following changes observable or measurable in a subject: a reduction in the number, duration or intensity of seizures or elimination of seizures; an improvement in cognitive function or memory; improved motor functions, or a reduction in the rate of motor function loss; an increase in sleep duration; prevention or reduction of neuronal cell death; reduce morbidity and mortality; improved quality of life; or a combination of such effects. In the case of pain, the therapeutically effective amount may cause a reduction in the frequency, duration and/or intensity of the pain, or eliminate the pain completely. As recognized by those skilled in the art, effective amounts may vary depending on route of administration, excipient usage, and co-usage with other agents.

The term “subject” refers to a living organism suffering from or prone to a condition that can be treated by administration of a compound or pharmaceutical composition as provided herein. Non-limiting examples include humans, other mammals and other non-mammalian animals.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Particular non-limiting examples of the present invention will now be described with reference to the following drawings, in which:

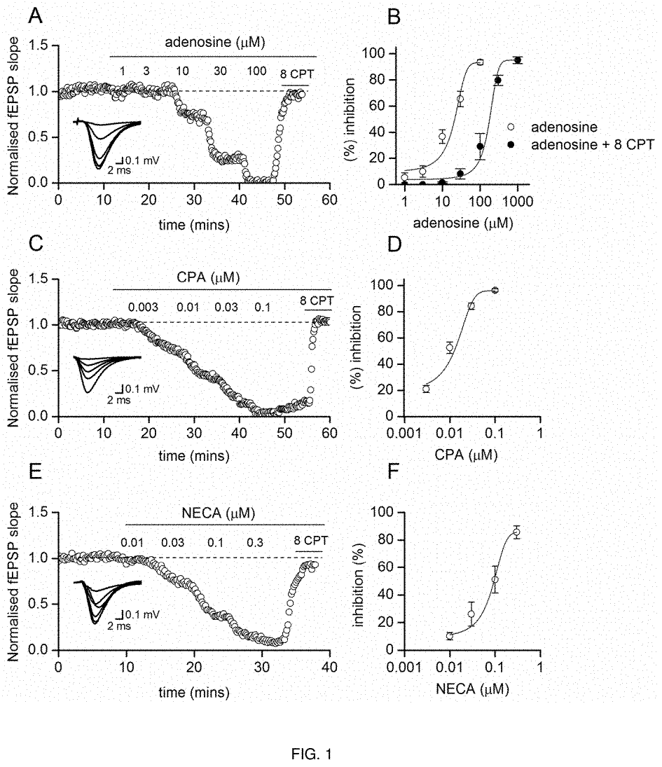

FIG. 1 shows the actions of prototypical adenosine receptor agonists on synaptic transmission in the hippocampus. A, Normalised fEPSP slope plotted against time for a single recording. Application of increasing concentrations of adenosine reduced fEPSP slope, an effect reversed by the A₁ receptor antagonist 8-CPT (2 μM). Inset, superimposed fEPSP averages in control and in increasing concentrations of adenosine. B, Concentration-response curve for adenosine (IC₅₀=20±4.3 μM, n=11 slices) and for adenosine with 2 μM 8-CPT (IC₅₀ n=5 slices). C, Normalised fEPSP slope plotted against time for a single recording. Application of increasing concentrations of the non-hydrolysable A₁ receptor agonist N⁶-CPA (CPA) reduced fEPSP slope, an effect reversed by the A₁ receptor antagonist 8-CPT (2 μM). Inset, superimposed fEPSP averages in control and in increasing concentrations of CPA. D, Concentration-response curve for CPA (IC₅₀=11.8±2.7 nM, n=11 slices). E, Normalised fEPSP slope plotted against time for a single recording. Application of increasing concentrations of the adenosine receptor agonist NECA reduced fEPSP slope, an effect reversed by the A₁ receptor antagonist 8-CPT (2 μM). Inset, superimposed fEPSP averages in control and in increasing concentrations of NECA. F, Concentration-response curve for NECA (IC₅₀=8.3±3 nM, n=11 slices).

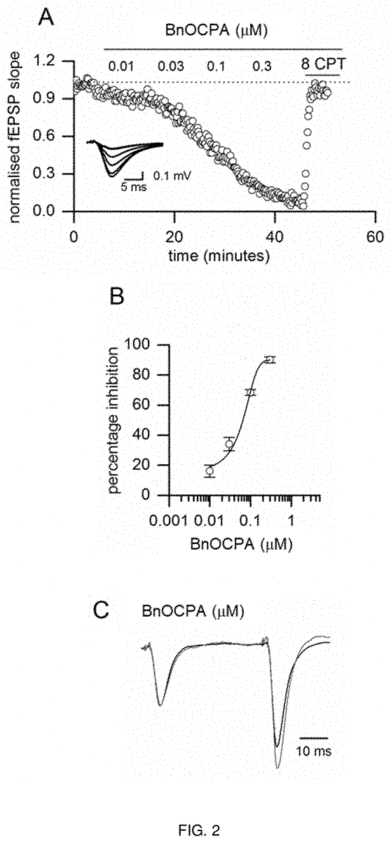

FIG. 2 shows the actions of atypical A₁ receptor agonist compound BnOCPA on synaptic transmission in the hippocampus. A, Example of data from a single experiment with normalised fEPSP slope plotted against time. Application of increasing concentrations of compound BnOCPA reduced fEPSP slope, an effect reversed by the A₁ receptor antagonist 8-CPT (4 μM). Inset, superimposed fEPSP averages in control and in increasing concentrations of compound BnOCPA. B, Concentration-response curve for compound BnOCPA (IC₅₀=65±0.3 nM, n=11 slices). C, Example of average (5 traces) superimposed paired-pulse fEPSP waveforms (50 ms interval between pulses) in control (black line) and in compound BnOCPA (grey line). The fEPSP traces have been normalised to the amplitude of the first fEPSP in control. For a paired-pulse interval of 50 ms, the paired-pulse ratio was significantly increased from 1.88±0.08 in control to 2.41±0.08 in BnOCPA (n=6 slices, P=0.005). The increase in the degree of paired-pulse facilitation is consistent with an action at presynaptic receptors.

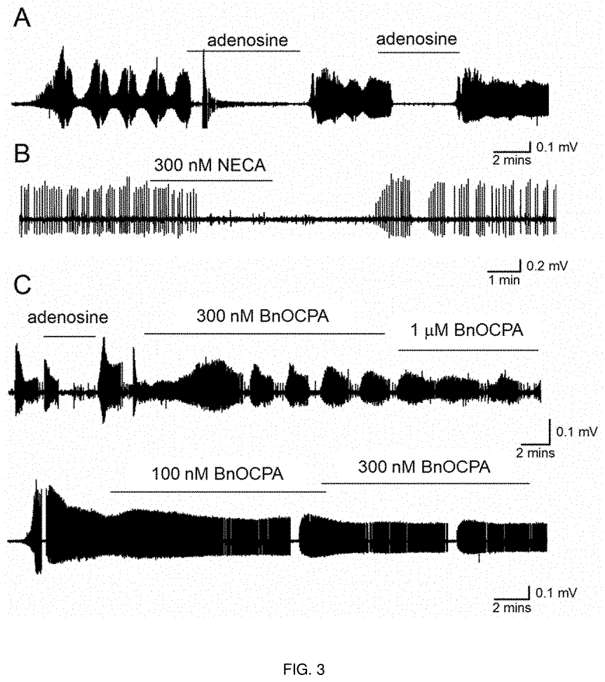

FIG. 3 shows the differential actions of adenosine receptor agonists on seizure activity. A, Example recording of seizure activity illustrating that adenosine (100 μM) reversibly blocks seizure activity. B, Example recording of seizure activity illustrating that NECA (300 nM) blocks seizure activity in a reversible manner. C, Example recordings of seizure activity from two different hippocampal slices illustrating that compound BnOCPA (300 nM to 1 μM) has little or no effect on seizure activity. Thus, BnOCPA has different actions on neural activity compared to prototypical adenosine A₁R agonists.

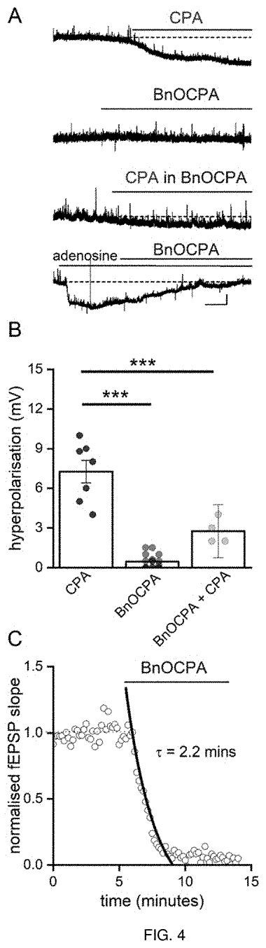

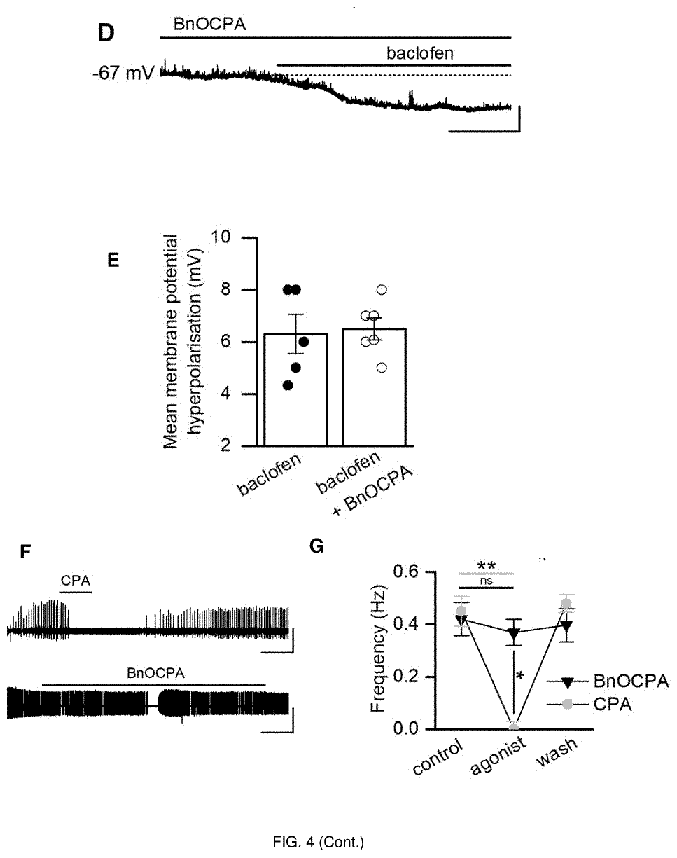

FIG. 4 shows the differential effects of proto- and the atypical adenosine receptor agonist BnOCPA on the membrane potential of pyramidal cells. A, Example traces of the membrane potential recorded from pyramidal cells in area CA1 of rat hippocampal slices. As expected, CPA hyperpolarised the membrane potential while in contrast, compound BnOCPA had no effect. Application of compound BnOCPA (300 nM) reduced the response to CPA (300 nM) and reversed the effects of adenosine (100 μM). The scale bar is 20 s for the top trace (CPA) and 40 s for the bottom two traces (compound BnOCPA and CPA+compound BnOCPA). B, Bar chart summarising the mean membrane potential hyperpolarisation (mV) produced by CPA (300 nM), compound BnOCPA (300 nM) and CPA (300 nM) in the presence of compound BnOCPA (300 nM). C, Graph plotting fEPSP slope against time for a single experiment. The same solution of compound BnOCPA used in (A) abolished synaptic transmission in a sister slice (the onset of inhibition is fitted with a single exponential τ=2.2 mins), confirming that compound BnOCPA was active during these studies. D, Application of baclofen (10 mM) in the presence of BnOCPA (300 nM) hyperpolarised the membrane potential (from −67 to −74 mV). Scale bars measure 5 mV and 50 s (CPA), 200 s (adenosine) or 100 s (baclofen). E, Data summary of baclofen/BnOCPA experiments. The mean hyperpolarisation produced by baclofen in the presence of BnOCPA was not significantly different (unpaired t-test) from that produced by baclofen in control conditions (6.5±0.43 mV vs 6.3±0.76 mV, P=0.774, n=5-6 cells for each condition). Bar chart displays individual data points and mean±SEM. F, In an in vitro model of seizure activity, represented as frequent spontaneous spiking from baseline, CPA (300 nM) reversibly blocked activity while BnOCPA (300 nM) had little effect. Scale bars measure 0.5 mV and 200 s. G, Summary data for seizure activity expressed in terms of the frequency of spontaneous spiking before, during and after CPA or BnOCPA. CPA abolished seizure activity (n=4) whereas BnOCPA did not significantly reduced seizure frequency (n=6) Data represented as mean±SEM; Two-way RM ANOVA (BnOCPA vs CPA slices): F(1, 3)=188.11, P=8.52×10⁻⁴ with the following Bonferroni post hoc comparisons: BnOCPA vs Control; P=1; CPA vs control; P=0.010; BnOCPA vs CPA; P=0.027. Averaged data is presented as mean±SEM. ns, not significant; *, P<0.05; **, P<0.02; ****, P<0.0001.

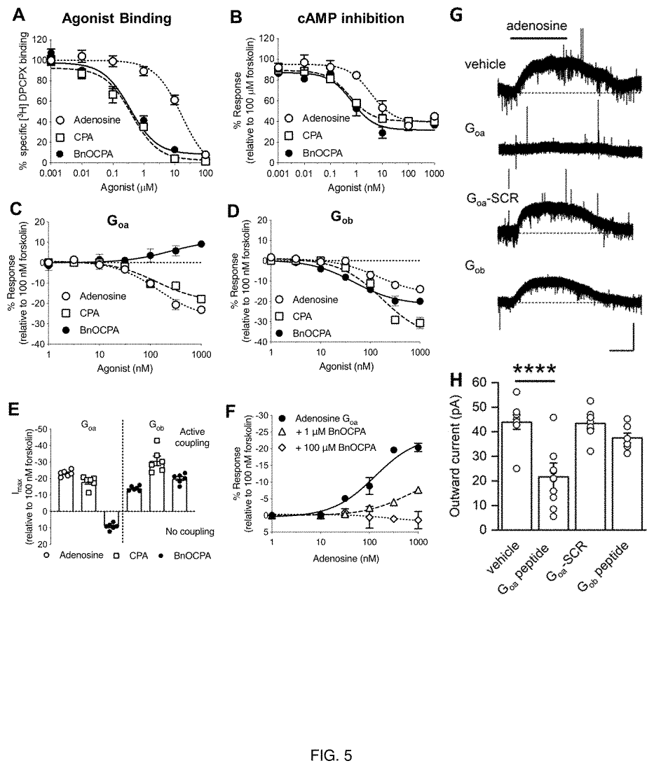

FIG. 5 shows the differential G protein activation profile of BnOCPA compared to prototypical A₁R agonists. A, The binding of adenosine, CPA and BnOCPA to the human (h) A₁R was measured via their ability to displace [³H]DPCPX, a selective antagonist for the A₁R, from membranes prepared from CHO-K1-hA₁R cells. The data indicate that CPA and BnOCPA bind with equal affinity to the A₁R, while adenosine has a reduced affinity (n=5-19 individual repeats). B, cAMP levels were measured in CHO-K1-hA₁R cells following co-stimulation with 1 μM forskolin and each compound (1 pM-1 μM) for 30 minutes. This identified that all are full agonists. Adenosine displays a 10-fold reduced potency compared to CPA and BnOCPA (n=4 individual repeats). C, cAMP accumulation was measured in PTX-pre-treated (200 ng/ml) CHO-K1-hA₁R cells expressing PTX-insensitive Goa following co-stimulation with 1 μM forskolin and each compound (1 pM-1 μM) for 30 minutes (n=6 individual repeats). The data demonstrates that BnOCPA does not activate Goa. D, as for (C), but cells were transfected with PTX-insensitive Gob. Adenosine, CPA and BnOCPA all inhibit cAMP accumulation through coupling to Gob (n=6 individual repeats). E, Summary of maximal A₁R-stimulated inhibition of cAMP by adenosine, CPA and BnOCPA in CHO-K1-hA₁R cells expressing either Goa (left) or Gob (right) obtained from data in panels C and D. F, Adenosine's ability to inhibit cAMP accumulation via its activation of Goa was inhibited by BnOCPA in a concentration-dependent manner and with a Kd of 113 nM (n=4 individual repeats). G, Example current traces produced by adenosine (10 μM) in control conditions, in the presence of intracellular Goa interfering peptide (100 μM), scrambled Goa peptide (SCR; 100 μM) and Gob interfering peptide (100 μM). Scale bars measure 50 pA and 100 s. H, Summary data of outward current experiments. The mean amplitude of the outward current induced by adenosine (43.9±3.1 pA, n=8 cells) was significantly reduced (one-way ANOVA; F(3,27)=13.31, P=1.60×10⁻⁵) to 20.9±3.6 pA (n=10 cells, P=5.35×10⁻⁵) in 100 μM Goa interfering peptide. Neither the scrambled peptide (43.4±2.4 pA, n=7 cells, P=1) nor the Gob interfering peptide (37.4±2.2 pA, n=6 cells, P=1) significantly reduced the amplitude of the adenosine-induced outward current. Averaged data is presented as mean±SEM. ****, P<0.0001.

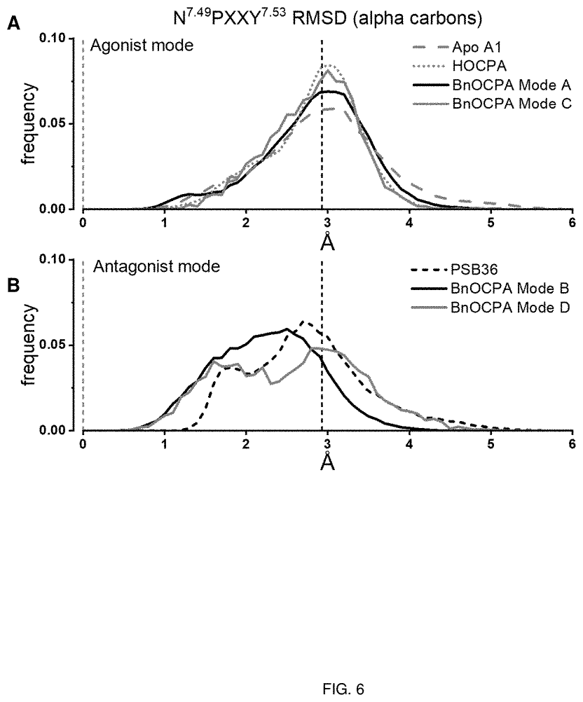

FIG. 6 shows root-mean-square deviation (RMSD) distributions considering the inactive N^{7.49}PXXY^{7.53} motif on the distal part of the TM7 as reference. A, HOCPA (dotted line), BnOCPA Mode A (solid black line), BnOCPA Mode C (solid grey line) and the apo receptor (dashed line) have a common distribution centring around the active confirmation of the A₁R (vertical black broken line), B, PSB36 (black dashed line), BnOCPA Mode B (solid black line) and BnOCPA Mode D (solid grey line) RMSD values have the tendency to move closer to the inactive N^{7.49}PXXY^{7.53} geometry (leftward shift of the curves towards broken grey line at x=0).

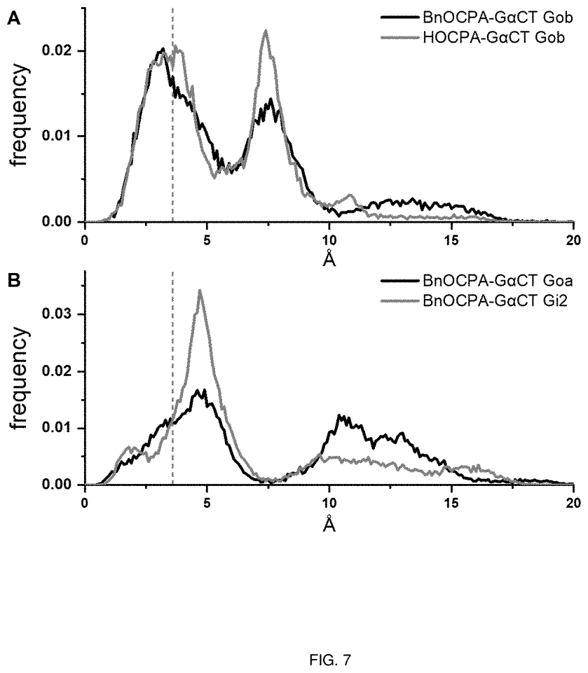

FIG. 7 shows plots of the frequency distribution of the RMSD of the last 15 residues of GαCT (alpha carbon atoms) to the Gi2 GαCT conformation reported in the A₁R cryo-EM structure 6D9H (the resolution of which, 3.6 Å, is indicated by the dashed vertical grey line). A, Dynamic docking of the Gob GαCT (last 27 residues) performed on the BnOCPA-A₁R (black line) and the HOCPA-A₁R (grey line) complex. The two most probable RMSD ranges, namely canonical state CS1 and metastable state MS1, can be observed. B, Dynamic docking of the Goa (black line) and Gi2 (grey line) GαCT (last 27 residues) performed on the BnOCPA-A₁R complex. The two most probable RMSD ranges are labelled as MS2 and MS3.

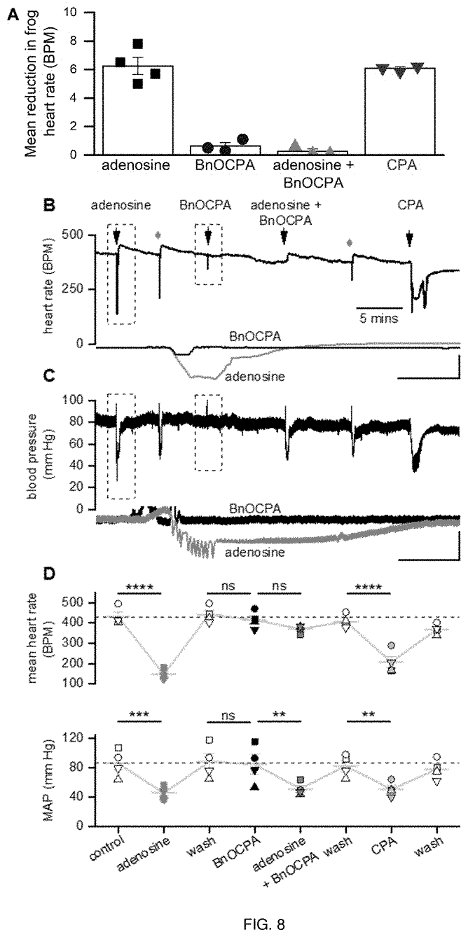

FIG. 8 shows the differential effects of BnOCPA compared to prototypical adenosine receptor agonists on heart rate and mean arterial pressure. A, Bar chart summarising the effects on isolated frog heart rate of adenosine (30 μM), BnOCPA (300 nM) and adenosine (30 μM) following compound BnOCPA application. In 4 preparations, adenosine reversibly reduced heart rate. Subsequent applications of compound BnOCPA had no significant effect on heart rate but reduced the effects of adenosine when it was applied again in the presence of BnOCPA. The prototypical A₁R synthetic agonist, CPA reduced the isolated frog heart rate similar to adenosine. To fully investigate the effects of BnOCPA on the CVS, its effects were measured on heart rate (HR) and mean arterial blood pressure (MAP) in urethane-anaesthetised, spontaneously breathing adult rats (FIG. 5B,C). B, Examples of heart rate (HR) and C, blood pressure (BP) traces from a single anaesthetised rat preparation and the effects of adenosine, BnOCPA and CPA. BnOCPA had no effect on HR or BP, in contrast to adenosine and CPA. The intravenous cannula was flushed at (*) to remove the compounds in the tubing. The overshoot in HR following adenosine applications is likely the result of the baroreflex. Insets, expanded HR and BP responses from boxed regions to adenosine (showing HR and BP depression) and to BnOCPA (no HR or BP depression). D, Summary data for 4 anaesthetised rat preparations. Data from each rat is shown as a different symbol. Data represented as mean±SEM. CPA and BnOCPA were given as a bolus at final doses about 300 and 500 times the IC₅₀ measured from their effects on synaptic transmission, respectively.

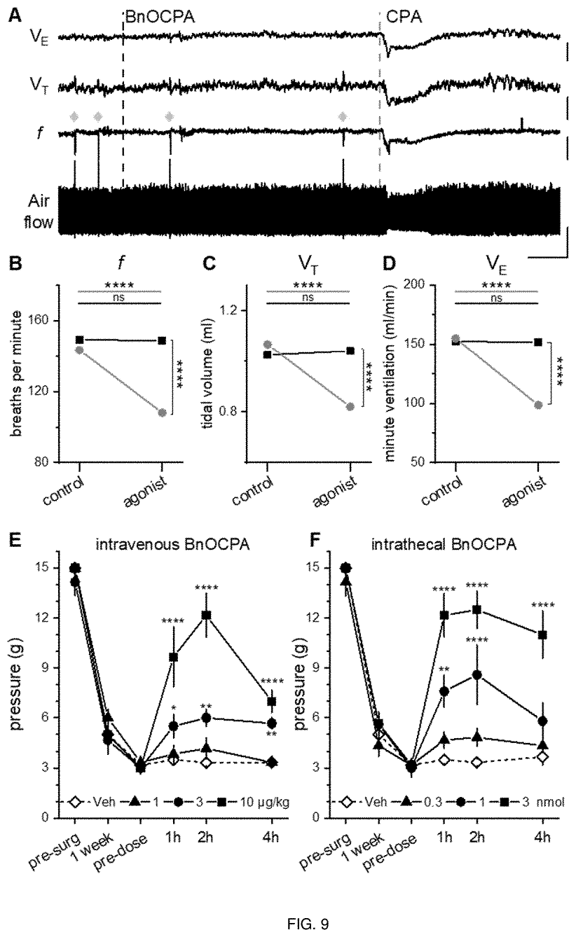

FIG. 9 shows that BnOCPA is a potent analgesic without causing respiratory depression. A, examples of tracheal airflow, respiratory frequency (f), tidal volume (V_{T}) and minute ventilation (V_{E}) from a single urethane-anaesthetised, spontaneously breathing rat showing the lack of effect of BnOCPA on respiration and the respiratory depression caused by CPA. BnOCPA and CPA were given as a 350 μL·kg⁻¹ IV bolus at the times indicated by the vertical broken lines (BnOCPA, 8.3 ug/kg; CPA, 6.3 μg·kg⁻¹). Grey diamonds indicate spontaneous sighs. Scale bars measure: 180 s and: airflow, 0.5 mL; f, 50 breaths per minute (BrPM); V_{T}, 0.25 mL; V_{E}, 50 mL/min. B, C, D, Summary data for 8 anaesthetised rats. Data from each rat is shown before and after the injection of BnOCPA (blue squares and broken lines) and CPA (red circles and broken lines) together with the mean value for all animals (solid lines) for f, V_{T} and V_{E}, respectively. One-way RM ANOVA: For: B, f, Greenhouse-Geisser corrected F(1.20, 8.38)=30.4, P=3.48×10⁻⁴; C, V_{T}, F(3, 21)=15.9, P=1.25×10⁻⁵, and D, V_{E}, Greenhouse-Geisser corrected F(1.19, 8.34)=15.77, P=0.003, with the following Bonferroni post hoc comparisons: Following BnOCPA, f (149±12 BrPM), V_{T} (1.0±0.1 mL), and V_{E} (152±26 ml/min) were not altered (P=1) compared to resting values f (149±12 BPM), V_{T} (1.0±0.1 mL), and V_{E} (152±26). In contrast to CPA, which reduced f (108±10 BrPM), V_{T} (0.8±0.1 mL), and V_{E} (99±19 ml/min) compared to resting values f (143±11 BrPM; p=4.05×10⁻⁶), V_{T} (1.1±0.1 mL; P=2.58×10⁻⁵), and V_{E} (155±28; P=5.52×10⁻⁵). Whilst the control resting values before administration of BnOCPA and CPA were not different to one another (P=1). The effects of CPA were significantly greater than BnOCPA for f (P=4.48×10⁻⁷), V_{T} (P=1.15×10⁻⁴), and V_{E} (P=1.16×10⁻⁴). Horizontal significance indicators above the data show differences between resting values and following IV administration of either BnOCPA (blue line) or CPA (red line). Vertical significance indicators show differences between the effects of BnOCPA and CPA. E, F, BnOCPA alleviates mechanical allodynia in a spinal nerve ligation (Chung) model of neuropathic pain when administered via an intravenous (IV; E) or intrathecal (IT; F) route. Prior to surgery (pre-surg) animals had similar sensitivity to tactile stimulation as assessed by Von Frey hair stimulation. Spinal nerve ligation subsequently caused hypersensitivity to touch (mechanical allodynia) as evidenced by the reduction in the tactile pressure necessary to elicit paw withdrawal (paw withdrawal threshold; PWT) at 1 week after surgery. PWT reaches a similar nadir across all groups prior to vehicle or BnOCPA infusion (pre-dose). Administration of BnOCPA significantly increased PWT in the limb ipsilateral to the site of injury, in a dose-dependent manner (one-way ANOVA (pre-dose, 1, 2 and 4 hrs) for IV BnOCPA: F(3,80)=37.3, P=3.44×10⁻¹⁵; for IT BnOCPA (3,76)=47.0, P=0). Fisher LSD post-hoc comparisons showed significant differences at: IV 3 ug/kg at 1, 2 and 4 hrs, P=0.044, 0.008 and 0.019, respectively, and 10 ug/kg at 1, 2 and 4 hrs, P=1.37×10⁻⁸, 6.81×10⁻¹⁴ and 3.23×10⁻⁴, respectively; IT 1 nmol at 1 and 2 hrs, P=0.001 and 4.16×10⁻⁵, respectively, and 10 nmol at 1, 2 and 4 hrs, P=9.52×10⁻¹¹, 1.42×10⁻¹¹ and 1.41×10⁻⁸, respectively. Averaged data (n=6 per treatment, except for 1 nmol BnOCPA, n=5) is presented as mean±SEM. ns, not significant; *, P<0.05; **, P<0.02; ***, P<0.001; ****, P<0.0001.

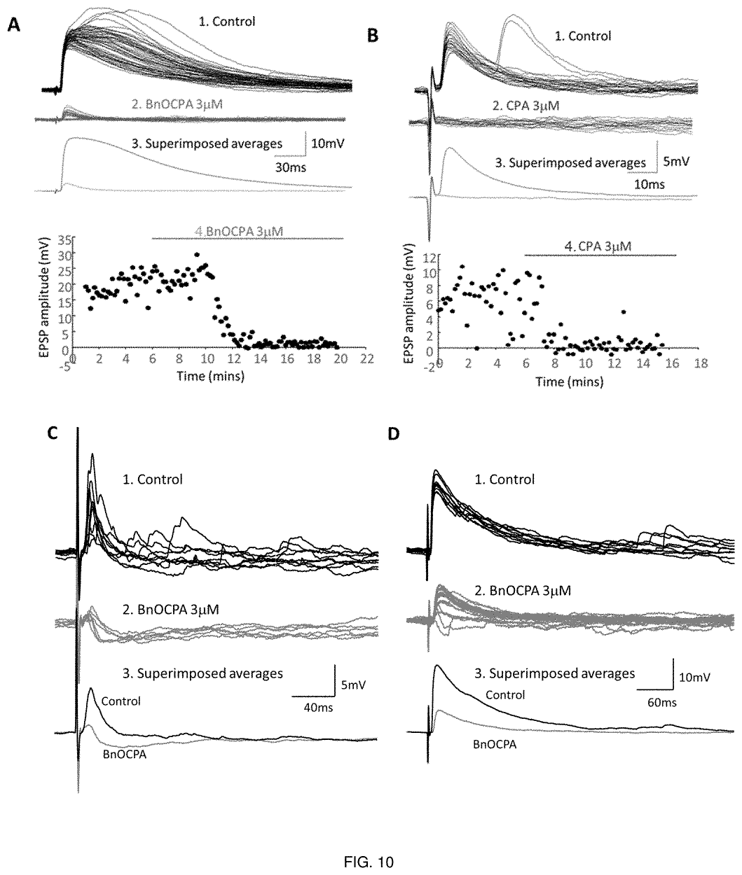

FIG. 10 shows the effects of BnOCPA and CPA on synaptic transmission in spinal nociceptive (pain sensing) afferents in rat (A, B) and non-human primate (Macaque: C, D) spinal cord. Samples of continuous records from 2 neurones (Left (A) and Right (B)) recorded in the dorsal horn of lumbar spinal cord slices prepared from adult rats. A, 1. Superimposed excitatory postsynaptic potentials (EPSPs) evoked by electrical stimulation of the dorsal roots at 0.1 Hz. A, 2. Same neurone showing EPSPs were reduced in the presence of compound BnOCPA. A, 3. Superimposed averages of EPSPs evoked in control and in the presence of compound BnOCPA. A, 4. Time-course plot showing the effects of compound BnOCPA on dorsal root afferent-evoked EPSPs. Note the significant reduction in amplitude of the EPSPs in compound BnOCPA. B, 1. Superimposed excitatory postsynaptic potentials (EPSPs) evoked by electrical stimulation of the dorsal roots at 0.1 Hz. B, 2. Same neurone showing EPSPs were reduced in the presence of prototypical A₁R agonist CPA. B, 3. Superimposed averages of EPSPs evoked in control and in the presence of CPA. B, 4. Time-course plot showing the effects of CPA on dorsal root afferent-evoked EPSPs. Note the significant reduction in amplitude of the EPSPs in CPA, comparable to that of BnOCPA. C, D, similar recordings made from neurons in lamina I/II dorsal horn of the lumbar spinal cord of the macaque. Samples of continuous records from two separate neurons (C and D) recorded in current clamp showing superimposed EPSPs in the absence (control) and presence (grey) of BnOCPA (3 μM). Bottom panel in each shows the superimposed averages of recordings in 1 and 2 for each cell.

### EXPERIMENTAL

Experiments were performed in accordance with the European Commission Directive 2010/63/EU (European Convention for the Protection of Vertebrate Animals used for Experimental and Other Scientific Purposes) and the United Kingdom Home Office (Scientific Procedures) Act (1986) with project approval from the institutional animal welfare and ethical review body (AWERB).

Preparation of hippocampal slices: Sagittal slices of hippocampus (300-400 μm) were prepared from male Sprague Dawley rats, at postnatal days 12 to 20. Rats were kept on a 12-hour light-dark cycle with slices made 90 minutes after entering the light cycle. In accordance with the U.K. Animals (Scientific Procedures) Act (1986), male rats were killed by cervical dislocation and decapitated. The brain was removed, cut down the mid line and the two sides of the brain stuck down to a metal base plate. Slices were cut around the midline with a Microm HM 650V microslicer in cold (2 to 4° C.) high Mg²⁺, low Ca2+ aCSF, composed of (mM): 127 NaCl, 1.9 KCl, 8 MgCl₂, 0.5 CaCl₂, 1.2 KH₂PO₄, 26 NaHCO₃, 10 D-glucose (pH 7.4 when bubbled with 95% O₂ and 5% CO₂, 300 mOSM). Slices were stored at 34° C. for 1 to 6 hours in aCSF (1 mM MgCl₂, 2 mM CaCl₂) before use.

Extracellular recording: A slice was transferred to the recording chamber, submerged in aCSF and perfused at 4 to 6 mL/min (32° C.). The slice was placed on a grid allowing perfusion above and below the tissue and all tubing was gas tight (to prevent loss of oxygen). For extracellular recording, an aCSF filled microelectrode was placed on the surface of stratum radiatum in CA1. Extracellular recordings were made using either a differential model 3000 amplifier (AM systems, WA USA) or a DP-301 differential amplifier (Warner Instruments, Hampden, Conn. USA) with field excitatory postsynaptic potentials (fEPSPs) evoked with either an isolated pulse stimulator model 2100 (AM Systems, WA) or ISO-Flex (AMPI, Jerusalem, Israel). For fEPSPs a 10 to 20 minute baseline was recorded at a stimulus intensity that gave 40 to 50% of the maximal response. Signals were filtered at 3 kHz and digitised on line (10 kHz) with a Micro CED (Mark 2) interface controlled by Spike software (Vs 6.1, Cambridge Electronic Design, Cambridge UK) or with WinLTP (W. W. Anderson et al., J. Neurosci Methods, 2007, 162, 346-356). For fEPSP slope, a 1 ms linear region after the fibre volley was measured. Extracellular recordings were made independently on two electrophysiology rigs. As the data obtained from each rig was comparable both sets of data have been pooled.

Seizure model: Seizure activity was induced in hippocampal slices using aCSF which contained no added Mg²⁺ and with the total K⁺ concentration increased to 6 mM with KCl. Removal of extracellular Mg²⁺ facilitates NMDA receptor activation producing long lasting EPSPS, which can sum together to produce tonic activation. Increasing the extracellular concentration of K⁺ depolarises neurons leading to firing and release of glutamate to sustain activity. Both the increase in K⁺ concentration and removal of Mg²⁺ are required to produce spontaneous activity in hippocampal slices. Spontaneous activity was measured with an aCSF-filled microelectrode placed within stratum radiatum in CA1.

Whole cell patch clamp recording from hippocampal pyramidal cells: A slice was transferred to the recording chamber and perfused at 3 mL/min with aCSF at 32±0.5° C. Slices were visualised using IR-DIC optics with an Olympus BX151W microscope (Scientifica) and a CCD camera (Hitachi). Whole-cell current clamp recordings were made from pyramidal cells in area CA1 of the hippocampus using patch pipettes (5 to 10 MΩ) manufactured from thick walled glass (Harvard Apparatus, Edenbridge UK) and containing (mM): potassium gluconate 135, NaCl 7, HEPES 10, EGTA 0.5, phosphocreatine 10, MgATP 2, NaGTP 0.3 and biocytin 1 mg/mL (290 mOSM, pH 7.2). Voltage and current recordings were obtained using an Axon Multiclamp 700B amplifier (Molecular Devices, USA) and digitised at 20 KHz. Data acquisition and analysis was performed using Pclamp 10 (Molecular Devices). For voltage clamp experiments, CA1 pyramidal cells were held at −60 mV. Peptides to interfere with G protein signalling were introduced via the patch pipette into the recorded cell. The cell was held for at least 10 minutes before adenosine (10 μM) was added to induce an outward current.

Frog heart preparation: Xenopus leavis frogs (young adult males) were supplied from Portsmouth Xenopus Resource Centre. Frogs were euthanized with MS222 (0.2% at a pH of 7), decapitated and pithed. The animals were dissected to reveal the heart and the pericardium carefully removed. Heart contractions were measured with a force transducer (AD instruments). Heart rate was acquired via a PowerLab 26T (AD instruments) controlled by LabChart 7 (AD instruments). The heart was regularly washed with ringer and drugs were applied directly to the heart.

In vivo anaesthetised rat preparation for cardiorespiratory recordings: Anaesthesia was induced in adult male Sprague Dawley rats (230-330 g) with isofluorane (2-4%; Piramal Healthcare). The femoral vein was catheterised for drug delivery. Anaesthesia was maintained with urethane (1.2-1.7 g/kg; Sigma) in sterile saline delivered via the femoral catheter. The femoral artery was catheterised and connected to a pressure transducer (Digitimer) to record arterial blood pressure. Body temperature was maintained at 36.7° C. via a thermocouple heating pad (TCAT 2-LV; Physitemp). The rats were then allowed to stabilise before the experiments began. Blood pressure signals were amplified using the NeuroLog system (Digitimer) connected to a 1401 interface and acquired on a computer using Spike2 software (Cambridge Electronic Design). Arterial blood pressure recordings were used to derive heart rate (HR: beats·minute⁻¹; BPM), and to calculate mean arterial blood pressure (MAP: Diastolic pressure+⅓*[Systolic Pressure−Diastolic pressure]). Airflow measurements were used to calculate: tidal volume (V_{T}; mL; pressure sensors were calibrated with a 3 mL syringe), and respiratory frequency (f; breaths·min-1; BrPM). Minute ventilation (V_{E}; mL·min-1) was calculated as f×V_{T}.

After allowing the animal to stabilise following surgery, A₁R agonists were administered by intravenous (IV) injection and the changes in HR, MAP, f, V_{T}, and V_{E} were measured. In pilot studies, the optimal dose of adenosine was determined by increasing the dose until robust and reliable changes in HR and MAP were produced (1 mg·kg⁻¹). The dose of CPA was adjusted until equivalent effects to adenosine were produced on HR and MAP (6.3 μg·kg⁻¹). For BnOCPA we initially used 5 μg·kg⁻¹, but saw no agonist effect on HR and MAP. To ensure this was not a false negative we increased the dose of BnOCPA (8.3 μg·kg⁻¹), which still gave no agonist effect on HR and MAP. However, as BnOCPA produced an antagonistic effect when co-administered with adenosine (FIG. 8), it must have reached A₁Rs at a high enough concentration to be physiologically active. These observations confirmed that the lack of agonistic effects on HR and MAP were not due to a type II error. 8.3 μg·kg⁻¹ BnOCPA was used for all further experiments. All injections were administered IV as a 350 μl·kg⁻¹ bolus.

In the experimental study, rats received an injection of adenosine. After cardiorespiratory parameters returned to baseline (5-10 minutes) rats were given BnOCPA. After allowing sufficient time for any effect of BnOCPA to be observed, rats received adenosine with BnOCPA co-administered in a single injection. After cardiorespiratory parameters returned to baseline, rats were injected with CPA.

To check that the volume of solution injected with each drug did not itself induce a baroreflex response leading to spurious changes in cardiorespiratory responses, equivalent volumes of saline (0.9%) were injected. These had no effect on either heart rate or MAP. To confirm that repeated doses of adenosine produced the same response and that the responses did not run-down, rats were given two injections of adenosine (1 mg·kg⁻¹). There was no significant difference in the changes in cardiovascular parameters produced by each adenosine injection.

An additional series of experiments (n=4) were undertaken to directly compare BnOCPA and CPA on respiration. Adult male Sprague Dawley rats (400-500 g) were anaesthetised with urethane and instrumented as described above, with the exception that the arterial cannulation was not performed.

After allowing the animal to stabilise following surgery, BnOCPA (8.3 μg·kg⁻¹) was administered. After a 20 minutes recovery period CPA (6.3 μg·kg⁻¹) was administered. All injections were administered IV as a 350 μl·kg⁻¹ bolus. Changes in f, V_{T}, and V_{E} were measured. If the dosing occurred close to a respiratory event such as a sigh a second IV dose was administered, with 20 minute recovery periods either side of the injection. Measurements for the effect of BnOCPA were time-matched to when CPA induced a change in respiration in the same preparation. As no difference was observed between the respiratory responses to BnOCPA in these rats (n=4) and those instrumented for both cardiovascular and respiratory recordings (n=4), the data were pooled (n=8; FIG. 9A to D).

Spinal cord slice preparation: Adult male Sprague-Dawley rats, aged 8-12 weeks (260-280 g), were housed in an air-conditioned room on a 12 hour light/dark cycle with food and water available ad libitum. Rats were terminally anaesthetized using isofluorane and decapitated. The vertebral column, rib cage and surrounding tissues was rapidly removed and pinned under ice-cold (<4° C.), high sucrose-containing aCSF of the following composition (mM): Sucrose 127, KCl 1.9, KH₂PO₄ 1.2, CaCl₂ 0.24, MgCl₂ 3.9, NaHCO₃ 26, D-glucose 10, ascorbic acid 0.5. A laminectomy was performed and the spinal cord and associated roots gently dissected and teased out of the spinal column and surrounding tissues. Dura and pia mater and ventral roots were subsequently removed with fine forceps and the spinal cord hemisected. Care was taken to ensure dorsal root inputs to the spinal cord were maintained. The hemisected spinal cord-dorsal root preparations were secured to a tissue slicer and spinal cord slices (400-450 μm thick) with dorsal roots attached cut in chilled (<4° C.) high sucrose aCSF using a Leica VT1000s microtome. Slices were transferred to a small beaker containing ice-cold standard aCSF (see below) and rapidly warmed to 35±1° C. in a temperature-controlled water bath over a 20 minute period, then subsequently removed and maintained at room temperature (22±2° C.) prior to electrophysiological recording. Slice incubation and electrophysiological recording aCSF was of the following composition (mM): NaCl 127, KCl 1.9, KH₂PO₄ 1.2, MgCl₂ 1.3, CaCl₂ 2.4, NaHCO₃ 26 and D-glucose 10. Similar procedures were adopted to make recordings from the macaque spinal cord following euthanasia by anaesthetic overdose.

Spinal cord electrophysiological recording: For electrophysiological recording, a spinal cord slice was transferred to a custom-built chamber. Connected slice and dorsal roots were continuously perfused with aCSF, at 35±1° C. at a flow rate of 5-10 mL·min⁻¹) to the slice and roots were maintained constant and consistent throughout recording. Whole-cell patch-clamp recordings were obtained from dorsal horn neurones of the spinal cord using Axopatch 1 D or 700 A amplifiers employing the “blind” version of the patch-clamp technique. Patch pipettes were pulled from thin-walled borosilicate glass with resistances of between 3 and 8 MΩ when filled with intracellular solution of the following composition (mM): K⁺gluconate, 140; KCl, 10; EGTA-Na, 1; HEPES, 10; Na₂ATP, 4, Na₂GTP, 0.3. Recordings were performed in the ‘current-clamp’ mode of the whole-cell patch clamp technique on slices continuously perfused with aCSF (rate: 4-10 mL/min; 35±1° C.). Drugs were administered to the slice by bath perfusion.

Electrical Stimulation of Dorsal Roots: Excitatory post-synaptic potentials (EPSPs) were evoked by electrical stimulation of the dorsal roots using a concentric bipolar stimulating electrode positioned on the roots. Control EPSPs were evoked at 0.1 Hz.

Drugs: Drugs were made up as stock solutions (1 to 10 mM) and then diluted in aCSF on the day of use. Compounds were dissolved in dimethyl-sulphoxide (DMSO, 0.01% final concentration of DMSO). Adenosine, 8-CPT (8-cyclopentyltheophylline), NECA (5′-(N-Ethylcarboxamido) adenosine) and CPA (N-Cyclopentyladenosine) were purchased from Sigma-Aldrich (Poole, Dorset, UK). BnOCPA was synthesised as previously published (Knight et al., J. Med. Chem., 2016, 59, 947-964). 1,3-[³H]-dipropyl-8-cyclopentylxanthine ([³H]-DPCPX) was purchased from PerkinElmer (Life and Analytical Sciences, Waltham, Mass.). Peptides for interfering with G protein signalling were obtained from Hello Bio (Bristol, UK) and were based on published sequences (Varani et al., Adv Exp Med Biol 1051, 193-232 (2017)). For Gₒₐ the peptide had a sequence of MGIANNLRGCGLY. The scrambled version was LNRGNAYLCIGMG. For G_{ob} the peptide had a sequence of MGIQNNLKYIGIC. Peptides were made up as stock solutions (2 mM) and stored at −20° C. The stock solutions were dissolved in filtered intracellular solution just before use.

Analysis: Concentration-response curves were constructed in OriginPro 2016 (OriginLab; Northampton, Mass., USA) and fitted with a logistic curve using the Levenberg Marquadt iteration algorithm. Statistical significance was tested using the unpaired t-test and one-way and two-way ANOVAs with Bonferroni correction for multiple comparisons.

Spinal nerve ligation (Chung model): Adult male Sprague-Dawley rats, 7-8 weeks old, weighing around 250 g at the time of Chung model surgery, were purchased from Charles River UK Ltd. The animals were housed in groups of 4 in an air-conditioned room on a 12-hour light/dark cycle. Food and water were available ad libitum. They were allowed to acclimatise to the experimental environment for three days by leaving them on a raised metal mesh for at least 40 min. The baseline paw withdrawal threshold (PWT) was examined using a series of graduated von Frey hairs (see below) for 3 consecutive days before surgery and re-assessed on the 6^{th} to 8^{th} day after surgery and on the 13^{th} to 17^{th} day after surgery before drug dosing.

Prior to surgery each rat was anaesthetized with 3% isoflurane mixed with oxygen (2 L·min⁻¹) followed by an i.m. injection of ketamine (60 mg·kg⁻¹) plus xylazine (10 mg·kg⁻¹). The back was shaved and sterilized with povidone-iodine. The animal was placed in a prone position and a para-medial incision was made on the skin covering the L4-6 level. The L5 spinal nerve was carefully isolated and tightly ligated with 6/0 silk suture. The wound was then closed in layers after a complete hemostasis. A single dose of antibiotics (Amoxipen, 15 mg/rat, i.p.) was routinely given for prevention of infection after surgery. The animals were placed in a temperature-controlled recovery chamber until fully awake before being returned to their home cages. The vehicle (normal saline) was administered via the intravenous (IV) route at 1 ml·kg⁻¹ and via the intrathecal (IT) route at 10 μl for each injection. The rats with validated neuropathic pain state were randomly divided into 8 groups: vehicle IV, BnOCPA at 1, 3, 10 μg·kg⁻¹ g IV; vehicle IT, BnOCPA at 0.3, 1, and 3 nmol IT groups.

To test for mechanical allodynia the animals were placed in individual Perspex boxes on a raised metal mesh for at least 40 minutes before the test. Starting from the filament of lower force, each filament was applied perpendicularly to the centre of the ventral surface of the paw until slightly bent for 6 seconds. If the animal withdrew or lifted the paw upon stimulation, then a hair with force immediately lower than that tested was used. If no response was observed, then a hair with force immediately higher was tested. The highest value was set at 15 g. The lowest amount of force required to induce reliable responses (positive in 3 out of 5 trials) was recorded as the value of PWT. On the testing day, PWT were assessed before and 1, 2 and 4 hours following BnOCPA or vehicle administration. The animals were returned to their home cages to rest (about 30 min) between two neighbouring testing time points. At the end of each experiment, the animals were deeply anaesthetised with isoflurane and killed by decapitation.

Rotarod test for motor function. A rotarod test was used to assess motor coordination following intravenous and intraperitoneal administration of BnOCPA. An accelerating rotarod (Ugo Basile) was set so speed increased from 6 to 80 rpm over 170 seconds. Male Sprague Dawley rats (n=24), 7 weeks of age (212-258 g) were trained on the rotarod twice daily for two days (≥2 trials per session) until performance times were stable. On the day of the experiment, three baseline trials were recorded. The compound was administered IP or intravenously via tail vein injection (10 μg/kg, n=6 per group). The control group received subcutaneous saline and the positive control group received subcutaneous morphine (15 mg/kg). Latency to fall (seconds) was measured in triplicate at 1, 2, 3 and 5 hours post drug administration.

Cell signaling assays. CHO-K1-hA₁R cells were routinely cultured in Hams F12 nutrient mix supplemented with 10% Foetal bovine serum (FBS), at 37° C. with 5% CO₂, in a humidified atmosphere. For cAMP inhibition experiments, cells were seeded at a density of 2000 cells per well of a white 384-well optiplate and co-stimulated, for 30 minutes, with 1 μM forskolin and a range of agonist concentrations (1 μM-1 pM). cAMP levels were then determined using a LANCE® cAMP kit.

For determination of individual Gα_{i/o/z} couplings, CHO-K1-hA₁R cells were transfected with pcDNA3.1-GNAZ or, pcDNA3.1 containing pertussis toxin (PTX) insensitive Gα_{i/o} protein mutants (C351I, C352I, C351I, C351I, C351I, for Gᵢ₁, Gᵢ₂, Gᵢ₃, Gₒₐ, G_{ob}, respectively, obtained from cDNA Resource Center; www.cdna.org), using 500 ng plasmid and Fugene HD at a 3:1 (Fugene:Plasmid) ratio. Cells were then incubated for 24 hours before addition of 100 ng/ml PTX, to inhibit activity of endogenous Gα_{i/o}, and then incubated for a further 16-18 hours. Transfected cells were then assayed as per cAMP inhibition experiments, but co-stimulated with agonist and 100 nM forskolin.

β-arrestin recruitment assays. HEK 293 cells were routinely grown in DMEM/F-12 GlutaMAX™ (Thermo Fisher Scientific) supplemented with 10% foetal bovine serum (FBS) (F9665, Sigma-Aldrich) and 1× antibiotic-antimycotic (Thermo Fisher Scientific) (DMEM complete). For analysis of β-arrestin2 recruitment following ligand stimulation at the human A₁R or A₃R, HEK 293 cells in a single well of 6-well plate (confluency≥80%) were transiently co-transfected with either A₁R-Nluc or A₃R-Nluc and β-arrestin2-YFP (total 2 μg, at a 1:6 ratio) using polyethyleneimine (PEI, 1 mg/ml, MW=25,000 g/mol) (Polysciences Inc) at a DNA:PEI ratio of 1:6 (w/v). Briefly, in sterile tubes containing 150 mM sodium chloride (NaCl), DNA or PEI was added (final volume 50 μl), allowed to incubate at room temperature for 5 minutes, mixing together and incubating for a further 10 minutes prior to adding the combined mix dropwise to the cells. 24 hours post-transfection, HEK 293 cell were harvested, resuspended in reduced serum media (MEM, NEAA (Thermo Fisher Scientific) supplemented with 1% L-glutamine (2 mM final) (Thermo Fisher Scientific), 2% FBS and 1× antibiotic-antimycotic) and seeded (50,000 cells/well) in a poly-L-lysine-coated (MW 150,000-300,000, Sigma-Aldrich) white 96-well plate (PerkinElmer Life Sciences). 24 hours post seeding, media was removed, cells gently washed in PBS and 90 μl of furimazine (4 μM) containing solution added (PBS supplemented with 0.49 mM MgCl₂, 0.9 mM CaCl₂ and 0.1% BSA) to each well before incubating in the dark for 10 minutes. After incubation, 10 μl of ligand (NECA/CPA, adenosine, BnOCPA) was added in the range of 10 μM to 0.01 μM and filtered light emission measured at 450 nm and 530 nm every minute for 1 hour using a Mithras LB 940 (Berthold technology). Here, Nluc on the C-terminus of A₁R or A₃R acted as the BRET donor (luciferase oxidizing its substrate) and YFP acted as the fluorescent acceptor. Vehicle control (DMSO) was added to determine background emission.

Radioligand binding. Radioligand displacement assays were conducted using crude membrane preparations (100 μg protein per tube) acquired from homogenisation of CHO-K1-hA₁R cells in ice-cold buffer (2 mM MgCl₂, 20 mM HEPES, pH 7.4). The ability to displace binding of the A₁R selective antagonist radioligand, 1,3-[³H]-dipropyl-8-cyclopentylxanthine ([³H]-DPCPX) at a concentration (1 nM) around the Kd value (1.23 nM, as determined by saturation binding experiments) by increasing concentrations of NECA, adenosine, CPA, BnOCPA or HOCPA (10 μM-0.1 nM) allowed the binding affinities (Ki) to be determined. Non-specific binding was determined in the presence of 10 μM DPCPX. Membrane incubations were conducted in Sterilin™ scintillation vials (Thermo Fisher Scientific; Wilmington, Mass., USA) for 60 minutes at room temperature. Free radioligand was separated from bound radioligand by filtration through Whatman® glass microfiber GF/B 25 mm filters (Sigma-Aldrich). Each filter was then placed in a Sterilin™ scintillation vial and radioactivity determined by: addition of 4 mL of Ultima Gold XR liquid scintillant (PerkinElmer), overnight incubation at room temperature and the retained radioactivity determined using a Beckman Coulter LS 6500 Multi-purpose scintillation counter (Beckman Coulter Inc.; Indiana, USA).

Data Analysis. Concentration-response curves for the effects of A₁R agonists on synaptic transmission were constructed in OriginPro 2018 (OriginLab; Northampton, Mass., USA) and fitted with a logistic curve using the Levenberg Marquadt iteration algorithm. OriginPro 2018 was also used for statistical analysis. Statistical significance was tested as indicated in the text using paired or unpaired t-tests or one-way or two-way ANOVAs with repeated measures (RM) as appropriate. Bonferroni corrections for multiple comparisons were performed. All in vitro cell signalling assay data was analysed using Prism 7.0e (Graphpad software, San Diego, Calif.), with all concentration-response curves being fitted using a 3 parameter logistic equation to calculate response range and IC₅₀. All cAMP data was normalised to a forskolin concentration-response curve ran in parallel to each assay. Statistical significance to adenosine was calculated using a one-way ANOVA with a Dunnett's post-test for multiple comparisons. Radioligand displacement curves were fitted to the one-site competition binding equation yielding log Ki values. One-way ANOVA (Dunnett's post-test) was used to determine significance by comparing the log Ki value for each compound when compared to adenosine. To determine the extent of ligand-induced recruitment of β-arrestin2-YFP to either the A₁R or A₃R, the BRET signal was calculated by subtracting the 530 nm/450 nm emission for vehicle-treated cells from ligand-treated cells (ligand-induced ΔBRET). ΔBRET for each concentration at 5 minutes (maximum response) was used to produce concentration-response curves.

All in vivo cardiovascular and respiratory data were analysed using OriginPro 2018. One-way ANOVAs, with repeated measures as appropriate, and with Bonferroni correction for multiple comparisons were used. Statistical significance for the effects of IV saline was tested using paired t-tests. Data are reported throughout as mean±SEM and n values are reported for each experiment. For the neuropathic pain studies, one-way ANOVAs with Fisher's Least Significant Difference (LSD) post-hoc test was used to compare drug treatment groups to the vehicle group (OriginPro 2018). The significance level was set at P<0.05, with actual P values reported in the figure legends and summaries, by way of abbreviations and asterisks, on the graphs: ns, not significant; * P<0.05; **, P<0.02; ***, P<0.001; ****, P<0.0001.

Molecular Dynamics Simulations

Ligand parameterization. The CHARMM36 (10, 11)/CGenFF (12-14) force field combination was employed in all the molecular dynamic (MD) simulations performed. Initial topology and parameter files of BnOCPA, HOCPA, and PSB36 were obtained from the Paramchem webserver. Higher penalties were associated with a few BnOCPA dihedral terms, which were therefore optimized at the HF/6-31G* level of theory using both the high throughput molecular dynamics (HTMD) parameterize functionality and the Visual Molecular Dynamics (VMD) Force Field Toolkit (ffTK) (17), after fragmentation of the molecule. Short MD simulations of BnOCPA in water were performed to visually inspect the behavior of the optimized rotatable bonds.

Systems preparation for fully dynamic docking of BnOCPA and HOCPA. Coordinates of the A₁R in the active, adenosine- and G protein-bound state were retrieved from the Protein Data Bank database (PDB ID 6D9H). Intracellular loop 3 (ICL3) which is missing from PDB ID 6D9H was rebuilt using Modeller 9.19. The G protein, with the exception of the C-terminal helix (helix 5) of the G protein alpha subunit (the key region responsible for the receptor TM6 active-like conformation) was removed from the system. BnOCPA and HOCPA were placed in the extracellular bulk, in two different systems, at least 20 Å from the receptor vestibule. The resulting systems were prepared for simulations using in-house scripts able to exploit both python HTMD and Tool Command Language (TCL) scripts. Briefly, this multistep procedure performs the preliminary hydrogen atoms addition by means of the pdb2pqr and propka software, considering a simulated pH of 7.0 (the proposed protonation of titratable side chains was checked by visual inspection). Receptors were then embedded in a square 80 Å×80 Å 1-palmitoyl-2-oleyl-sn-glycerol-3-phosphocholine (POPC) bilayer (previously built by using the VMD Membrane Builder plugin 1.1, Membrane Plugin, Version 1.1.; http://www.ks.uiuc.edu/Research/vmd/plugins/membrane/) through an insertion method, considering the A₁R coordinates retrieved from the OPM database to gain the correct orientation within the membrane. Lipids overlapping the receptor transmembrane bundle were removed and TIP3P water molecules were added to the simulation box (final dimensions 80 Å×80 Å×125 Å) using the VMD Solvate plugin 1.5 (Solvate Plugin, Version 1.5; http://www.ks.uiuc.edu/Research/vmd/plugins/solvate/). Finally, overall charge neutrality was achieved by adding Na⁺/Cl⁻ counter ions (concentration of 0.150 M) using the VMD Autoionize plugin 1.3 (Autoionize Plugin, Version 1.3; http://www.ks.uiuc.edu/Research/vmd/plugins/autoionize/). All histidine side chains were considered in the delta tautomeric state, with the exception of H251 (epsilon tautomer) and H278 (protonated).

The MD engine ACEMD was employed for both the equilibration and productive simulations. Systems were equilibrated in isothermal-isobaric conditions (NPT) using the Berendsen barostat (31) (target pressure 1 atm), the Langevin thermostat (target temperature 300 K) with a low damping factor of 1 ps⁻¹ and with an integration time step of 2 fs. Clashes between protein and lipid atoms were reduced through 2000 conjugate-gradient minimization steps before a 2 ns long MD simulation was run with a positional constraint of 1 kcal mol⁻¹ Å⁻² on protein and lipid phosphorus atoms. Twenty nanoseconds of MD simulation were then performed constraining only the protein atoms. Lastly, positional constraints were applied only to the protein backbone alpha carbons for a further 5 ns.

Dynamic docking of BnOCPA and HOCPA. The supervised MD (SuMD) approach is an adaptive sampling method for simulating binding events in a timescale one or two orders of magnitudes faster than the corresponding classical (unsupervised) MD simulations. In the present work, the distances between the centers of mass of the adenine scaffold of the A₁R agonist and N254^{6.55}, F171^{ECL2}, T277^{7.42} and H278^{7.43} of the receptor were considered for the supervision during the MD simulations. The dynamic docking of BnOCPA was hindered by the ionic bridge formed between the E172^{ECL2} and K265^{ECL3} side chains. A metadynamics energetic bias was therefore introduced in order to facilitate the rupture of this ionic interaction, thus favouring the formation of a bound complex. More precisely, Gaussian terms (height=0.01 kcal mol-1 and widths=0.1 Å) were deposited every 1 ps along the distance between the E172^{ECL2} carboxyl carbon and the positively charged K265^{ECL3} nitrogen atom using PLUMED 2.3. For each replica, when the ligands reached a bound pose (i.e. a distance between the adenine and the receptor residues centers of mass <3 Å), a classic (unsupervised and without energetic bias) MD simulation was performed for at least a further 100 ns.

BnOCPA bound state metadynamics. We decided to perform a detailed analysis of the role played by the E172^{ECL2}-K265^{ECL3} ionic interaction in the dynamic docking of BnOCPA. Three 250 ns long well-tempered metadynamics simulations were performed using the bound state obtained from a previous dynamic docking simulation, which resulted in binding mode A, as a starting point. The collective variables (CVs) considered were: i) the distance between the E172^{ECL2} carboxyl carbon and the positively charged K265^{ECL3} nitrogen atom and ii) the dihedral angle formed by the 4 atoms linking the cyclopentyl ring to the phenyl moiety (which was the most flexible ligand torsion during the previous SuMD simulations). Gaussian widths were set at 0.1 Å and 0.01 radians respectively, heights at 0.01 kcal/mol⁻¹, and the deposition was performed every 1 ps (bias-factor=5). Three replicas allowed sampling of three main energetic minima on the energy surface.

Classic MD simulations of BnOCPA binding modes A, B, C and D. To test the hypothesis that BnOCPA and HOCPA may differently affect TM6 and/or TM7 mobility when bound to A₁R (and to further sample the stability of each BnOCPA binding mode), putative binding conformations A, B and C were superposed to the experimental A₁R active state coordinates with the modelled ICL3. This should have removed any A₁R structural artefacts, possibly introduced by metadynamics. As reference and control, two further systems were considered: i) the pseudo-apo A₁R and ii) the selective A₁R antagonist PSB36 superposed in the same receptor active conformation. The BnOCPA binding mode D was modelled from mode B by rotating the dihedral angle connecting the cyclopentyl ring and the N6 nitrogen atom in order to point the benzyl of the agonist toward the hydrophobic pocket underneath ECL3 delimited by L253^{6.56}, T257^{6.52}, K265^{ECL3}, T270^{7.35}, and L269^{7.34}. The G protein atoms were removed, and the resulting systems prepared for MD as reported above.

Dynamic docking of the Goa, Gob and Gi2 GαCT helix. A randomly extracted frame from the classic MD performed on the BnOCPA:A1R complex was prepared for three sets of simulations placing the GαCT helix 5 (last 27 residues) of the Gα proteins Goa, Gob and Gi2 in the intracellular solvent bulk side of the simulation boxes. As a further control, a frame from the classic MD performed on the unbiased ligand HOCPA:A₁R complex was randomly extracted and prepared along with the Gob GαCT. The resulting four systems were embedded in a POPC membrane and prepared as reported above.

The different structural effects putatively triggered by BnOCPA and HOCPA on the recognition mechanism of Goa, Gob and Gi2 GαCT were studied by performing 10 SuMD replicas. During each replica, the distance between the centroid of the GαCT residues 348-352 and the centroid of the A1R residues D42^{2.37}, I232^{6.33}, and Q293^{8.48} was supervised until it reached a value lower than 8 Å. A classic MD simulation was then run for a further 300 ns.

Classic MD simulations on the A₁R:Goa and Gob complexes. The A₁R cryo-EM structure (PDB ID 6D9H) was used as template for all the five systems simulated. The endogenous agonist adenosine was removed and HOCPA and BnOCPA (modes B and D) were inserted in the orthosteric site superimposing 6D9H to the systems prepared for the classic MD simulations in the absence of G protein. ICL3 was not modelled, nor were the missing part of the G protein a subunit. As subunits β and γ were removed, the Gα NT helix was truncated to residue 27 to avoid unnatural movements (NT is constrained by Gβ in 6D9H). The Gα subunit was mutated according to the Goa and Gob primary sequences using in-house scripts. The resulting five systems were embedded in a POPC membrane and prepared as reported above.

Analysis of the classic MD simulations. During the classic MD simulations that started from Modes A-C, BnOCPA had the tendency to explore the three conformations by rapidly interchanging between the three binding modes. In order to determine the effect exerted on the TM domain by each conformation, 21 μs of MD simulations (BnOCPA mode A, BnOCPA mode B, BnOCPA mode C) were subjected to a geometric clustering. More precisely, a simulation frame was considered in pose A if the distance between the phenyl ring of BnOCPA and the I175^{ECL2} alpha carbon was less than 5 Å; in pose B if the distance between the phenyl ring of BnOCPA and the L258^{6.59} alpha carbon was less than 6 Å, and in pose C if the distance between the phenyl ring of BnOCPA and the Y271^{7.36} alpha carbon was less than 6 Å. During the MD simulations started from mode D, a frame was still considered in mode D if the root mean square deviation (RMSD) of the benzyl ring to the starting equilibrated conformation was less than 3 Å. For each of the resulting four clusters, the RMSD of the GPCR conserved motif NPXXY (N^{7.49} PIV Y^{7.53} in the A₁R) was computed using Plumed 2.3 considering the inactive receptor state as reference, plotting the obtained values as frequency distributions (FIG. 6). Rearrangement of the NPXXY motif, which is located at the intracellular half of TM7, is considered one of the structural hallmarks of GPCR activation. Upon G protein binding, it moves towards the center of the Receptor™ bundle. Unlike other activation micro-switches (e.g. the break/formation of the salt bridge between R^{3.50} and E^{6.30}), this conformational transition is believed to occur in timescales accessible to MD simulations.

Hydrogen bonds and atomic contacts were computed using the GetContacts analysis tool (https://github.com/getcontacts/getcontacts) and expressed in terms of occupancy (the percentage of MD frames in which the interaction occurred).

Analysis of the Goa, Gob and Gi2 GαCT classic MD simulations after SuMD. For each system, only the classic MD simulations performed after the GαCT reached the A1R intracellular binding site were considered for the analysis.

The RMSD values to the last 15 residues of the Gi2 GαCT reported in the A₁R cryo-EM PDB structure 6D9H were computed using VMD. The MD frames associated with the peaks in the RMSD plots (states CS1, MS1, MS2 and MS3 in FIG. 7A, D) were clustered employing the VMD Clustering plugin (https://github.com/luisico/clustering) by selecting the whole GαCT helixes alpha carbon atoms and a cutoff of 3 Å.

### Example 1: Synthesis of Cyclopentyladenosine (CPA) Derivatives—General Procedures

tBuOCPA was prepared according to the following procedure:

### (i) tert-butyl ((1R,2R)-2-hydroxycyclopentyl)carbamate (1)

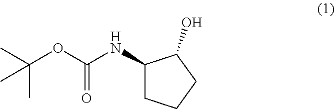

(1R,2R)-2-aminocyclopentanol hydrochloride (1 eq, 10.9 mmol) was dissolved in 70 mL dichloromethane and di-tert-butyl decarbonate (1 eq, 10.9 mmol) was added. The suspension was stirred at room temperature. To the suspension, N,N-diisopropylethylamine (1 eq, 10.9 mmol) was added. After 2 hours, the clear solution was concentrated under reduced pressure. After purification by silica gel chromatography (hexane/ethyl acetate gradient) 1 was obtained as a white solid (2.03 g, 10.3731 mmol, 93%). ¹H NMR: (300 MHz, DMSO-d₆) δ 6.71 (d, J=7.4 Hz, 1H), 4.60 (d, J=4.3 Hz, 1H), 3.77 (m, 1H), 3.49 (m, 1H), 1.95-1.68 (m, 2H), 1.61 (m, 2H), 1.42-1.24 (m, 1H), 1.38 (s, 9H), 1.37-1.24 (m, 2H). ¹³C NMR: (75 MHz, DMSO-d₆) δ 155.73, 77.84, 76.49, 59.20, 32.53, 30.08, 28.75, 20.83. HR-MS: (NSI+), ACN, [M+H]⁺: m/z calculated 224.1250, found 224.1257, Δ: −3.41 ppm.

### (ii) tert-butyl ((1R,2R)-2-((4-(tert-butyl)benzyl)oxy)cyclopentyl)carbamate (2)

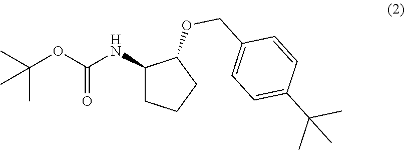 [SMILES: CC(C)(C)OC(=O)N[C@@H]1CCC[C@H]1OCc1ccc(C(C)(C)C)cc1]

Compound 1 (1 eq, 1.242 mmol) and 4-tert-butylbenzyl bromide (1 eq, 1.242 mmol) were dissolved in dry THF. The reaction mixture was cooled to 0° C. and NaH 60% dispersion in mineral oil (2 eq, 2.484 mmol) was added. After 1 hour and 30 minutes at 0° C., methanol (0.1 mL) and NH₄Cl aq. were added and the flask was removed from the ice bath. The reaction mixture was extracted with ethyl acetate, the organic phase was dried over sodium sulfate and concentrated under reduced pressure. After purification by silica gel chromatography (hexane/ethyl acetate gradient), 2 was obtained as an oil (126 mg, 0.363 mmol, 30%). ¹H NMR: (300 MHz, DMSO-d₆) δ 7.35 (d, J=8.2 Hz, 2H), 7.22 (d, J=8.2 Hz, 2H), 6.93-6.84 (m, 1H), 4.51-4.40 (m, 2H), 3.75 (s, 1H), 3.69 (m, 1H), 1.94-1.72 (m, 2H), 1.65-1.51 (m, 3H), 1.40 (m, 1 h) 1.40 (s, 9H), 1.27 (s, 9H). ¹³C NMR: (101 MHz, DMSO) δ 155.44, 150.09, 136.33, 127.77, 125.33, 84.81, 78.01, 70.08, 56.89, 34.66, 31.63, 30.65, 30.55, 28.76, 21.88. HR-MS: (NSI+), ACN, [M+H]⁺: m/z calculated 348.2533, found 348.2533, Δ: 0.03 ppm.

### (iii) (1R,2R)-2-((4-(tert-butyl)benzyl)oxy)cyclopentan-1-aminium chloride (3)

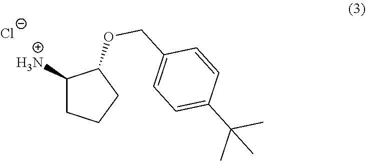 [SMILES: [Cl-] ; CC(C)(C)c1ccc(CO[C@@H]2CCC[C@H]2[NH3+])cc1]

Compound 2 (1 eq, 0.329 mmol) was dissolved in 1 mL dioxane and HCl in dioxane 4N (5 eq, 1.649 mmol) was added. After 5 hours the solvent was removed under reduced pressure. After co-evaporation with dichloromethane, 3 was obtained as a white solid (93 mg, 0.328 mmol, 99%). ¹H NMR: (300 MHz, DMSO-d₆) δ 8.02 (s, 2H), 7.42-7.35 (m, 2H), 7.28 (d, J=8.3 Hz, 2H), 4.54-4.39 (m, 2H), 3.93-3.86 (m, 1H), 3.42 (s, 1H), 2.00 (m, 2H), 1.75-1.48 (m, 5H), 1.28 (s, 9H). ¹³C NMR: (75 MHz, DMSO-d6) δ 150.35, 135.74, 128.01, 125.38, 82.82, 70.70, 56.29, 34.70, 31.63, 30.27, 28.92, 21.64. HR-MS: (NSI+), ACN, [M+H]⁺: m/z calculated 248.2005, found 248.2009, Δ: −1.70 ppm.

### (iv)(2R,3R,4R,5R)-2-(acetoxymethyl)-5-(6-(((1S,2R)-2-((4-(tert-butyl)benzyl)oxy)cyclopentyl)amino)-9H-purin-9-yl)tetrahydrofuran-3,4-diyl diacetate (4)

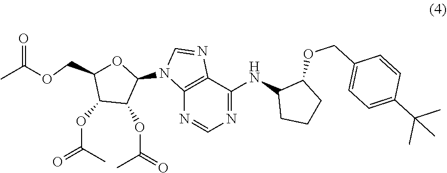

(2R,3R,4R,5R)-2-(acetoxymethyl)-5-(6-chloro-9H-purin-9-yl)tetrahydrofuran-3,4-diyl diacetate (1 eq, 0.235 mmol) was dissolved in 15 mL isopropanol. NaHCO₃ (3 eq, 0.705 mmol) and 3 (1.5 eq, 0.352 mmol) were added. The reaction mixture was heated at 105° C. under reflux overnight. At reaction completion, the reaction mixture was let to cool down until room temperature and the remaining solid was filtered off and washed with absolute ethanol. The filtrate was evaporated under reduced pressure. After purification by silica gel chromatography (hexane/ethyl acetate gradient), 4 was obtained as a solid (52.7 mg, 0.0845 mmol, 36%). 1H NMR: (300 MHz, Methanol-d₄) δ 8.31 (s, 1H), 8.24 (s, 1H), 7.32 (d, J=8.5 Hz, 2H), 7.23 (d, J=8.4 Hz, 2H), 6.25 (d, J=5.3 Hz, 1H), 6.03 (t, J=5.5 Hz, 1H), 5.73 (dd, J=5.7, 4.5 Hz, 1H), 4.67 (s, 1H), 4.63 (s, 2H), 4.50-4.35 (m, 3H), 4.01 (m, 1H), 2.26 (m, 1H), 2.16 (s, 3H), 2.08 (d, J=1.8 Hz, 6H), 2.05-1.99 (m, 1H), 1.90-1.57 (m, 5H), 1.29 (s, 9H). ¹³C NMR: (101 MHz, Methanol-d₄) δ 170.80, 169.98, 169.74, 154.33, 152.85, 139.22, 135.53, 127.34, 124.73, 86.39, 84.68, 80.21, 72.99, 70.74, 70.61, 62.83, 33.90, 30.39, 30.26, 30.03, 21.11, 19.24, 19.04, 18.87. HR-MS: (NSI+), ACN, [M+H]⁺: m/z calculated 624.3012, found 624.3028, Δ: −2.53 ppm.

### (v) (2R,3R,4S,5R)-2-(6-(((1S,2R)-2-((4-(tert-butyl)benzyl)oxy)cyclopentyl)amino)-9H-purin-9-yl)-5-(hydroxymethyl)tetrahydrofuran-3,4-diol (tBuOCPA)

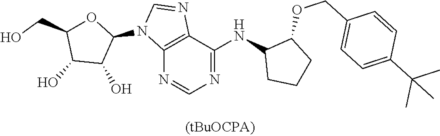

Compound 4 (1 eq, 0.0834 mmol) was dissolved in 4 mL methanol and K₂CO₃ (0.6 eq, 0.0500 mmol) was added at room temperature. After 30 minutes the reaction mixture was filtered and concentrated under reduced pressure. After purification by silica gel column chromatography, tBuOCPA was obtained as a white solid (30 mg, 0.0603 mmol, 73%). 1H NMR: 300 MHz, Methanol-d4) δ 8.16 (s, 2H), 7.21 (d, J=8.4 Hz, 2H), 7.11 (d, J=8.3 Hz, 2H), 5.85 (d, J=6.5 Hz, 1H), 4.65 (dd, J=6.5, 5.1 Hz, 1H), 4.57 (s, 1H), 4.40-4.55 (m, 1H), 4.22 (dd, J=5.1, 2.5 Hz, 1H), 4.06-4.09 (m, 1H), 3.87-3.91 (m, 4.0 Hz, 1H), 3.79 (dd, J=12.6, 2.5 Hz, 1H), 3.64 (dd, J=12.5, 2.6 Hz, 1H), 2.09-2.20 (m, 1H), 1.99-1.85 (m, 1H), 1.80-1.45 (m, 4H), 1.18 (s, 9H). ¹³C NMR: (101 MHz, Methanol-d₄) δ 154.46, 152.15, 150.22, 140.05, 135.50, 127.33, 124.74, 89.95, 86.85, 84.58, 74.06, 71.32, 70.70, 62.13, 33.91, 30.38, 30.19, 29.99, 21.09. UPLC: t_{R}=3.01 min. (NSI+), ACN, [M+H]⁺: m/z calculated 498.2690, found 498.2711, Δ: −4.21 ppm.

It will be appreciated that the synthetic methods above can be adapted by the skilled person to prepare other compounds according to the invention, for example by using alternative electrophiles for the alkylation of the oxygen in step (ii).

### Example 2: Effect of Compound BnOCPA on Synaptic Transmission in Rat Hippocampus

In order to assess the action of the A₁ receptor agonist compound BnOCPA in mammalian brain tissue, firstly its effects on excitatory synaptic transmission, which is strongly inhibited by the activation of adenosine A₁ receptors on presynaptic terminals, was investigated. The effects of the agonist were compared to the effects of established adenosine receptor agonists (adenosine, CPA and NECA). Synaptic transmission was inhibited by the endogenous non-specific agonist adenosine (FIG. 1A) with an IC₅₀ (the half maximal inhibitory concentration) of 20±4.3 μM (n=11 slices, FIG. 1B). Application of the specific adenosine A₁ receptor antagonist 8-CPT (2 μM), which reversed the inhibition of synaptic transmission caused by adenosine (FIG. 1A), produced a parallel and rightward shift in the adenosine concentration-response curve (IC₅₀ shifted from 20±4.3 μM to 125±10 μM, n=5; FIG. 1B). These data confirm that the inhibitory effects of adenosine were via the activation of A₁ receptors. Both CPA and NECA inhibited synaptic transmission, in a manner that was reversed by 8-CPT, with IC₅₀s of 11.8±2.7 nM (n=11 slices, FIG. 1C, D) and 8.3±3 nM (n=11 slices; FIG. 1E, F), respectively.

Compound BnOCPA inhibited synaptic transmission, in a manner that was reversed by co-application of the A₁ receptor antagonist 8-CPT (4 μM, n=12; FIG. 2). From the concentration-response curves, the IC₅₀ for compound BnOCPA was calculated as 65±0.3 nM; n=11 slices (FIG. 2B).

To establish if this inhibition of synaptic transmission was presynaptic in nature, paired-pulse facilitation experiments were performed, in which the paired-pulse ratio is inversely proportional to the initial probability of transmitter release. Thus, compounds that inhibit synaptic transmission by reducing transmitter release would be expected to increase paired-pulse facilitation. Compound BnOCPA (100 nM) significantly increased paired-pulse facilitation (for a paired-pulse interval of 50 ms the paired-pulse ratio increased from 1.88±0.08 in control slices to 2.41±0.08; n=6 slices, FIG. 2C). Thus, the action of compound BnOCPA is consistent with the activation of presynaptic A₁ receptors to inhibit synaptic transmission in the hippocampus.

### Example 3: Effects of Compound BnOCPA on Seizure Activity in Hippocampal Slices

The actions of the atypical agonist compound BnOCPA on seizure activity induced in hippocampal slices were investigated. During seizure activity, adenosine is released to activate A₁ receptors leading to termination of the burst of activity, and also delaying the occurrence of the next burst. Application of exogenous adenosine and other A₁ receptor ligands would therefore be expected to inhibit activity (see, for example: M. J. Wall et al., 2015). The actions of compound BnOCPA was compared to the actions of prototypical agonists. A nominally Mg²⁺-free/increased K⁺ (6 mM) aCSF was used to initiate seizure activity in the hippocampus, reflected by the appearance of robust long-lasting epileptiform activity characterised by frequent neuronal spikes (see, for example: J. Lopatář et al., Neuropharmacology, 2011, 61, 25-34). Adenosine (20 to 100 μM, n=5 slices; FIG. 3A) and NECA (300 nM, n=4 slices; FIG. 3B) rapidly and reversibly abolished seizure activity which recovered after washout of the agonist. 0.3 to 1 μM (i.e. about 5-15 times the IC₅₀ against synaptic transmission, 65 nM) of compound BnOCPA did not abolish seizure activity and appeared to have little effect on either burst frequency or duration (n=6 slices; FIG. 3C). This was a surprising result considering that compound BnOCPA strongly inhibited synaptic transmission (see Example 2).

There are two components to the anti-seizure effects of A₁ receptor agonists: presynaptic inhibition of excitatory synaptic transmission, and the postsynaptic hyperpolarisation of the neuronal membrane potential. It is hypothesised that the weak effect of compound BnOCPA against seizure activity arose from an inability to hyperpolarise the postsynaptic membrane potential, unlike other prototypical A₁ receptor agonists.

### Example 4: Effects of Compound BnOCPA on Membrane Potential Hyperpolarisation in Pyramidal Cells

To establish the influence of postsynaptic A₁ receptor activation, the degree of membrane potential hyperpolarisation in CA1 pyramidal cells produced via the activation of K⁺ channels was measured. Adenosine (100 μM) and CPA (300 nM) markedly hyperpolarised the membrane potential of pyramidal cells (adenosine: membrane potential changed from −69.4±1.5 to −74.1±1.5 mV, mean change of −4.7±0.5 mV, n=8; CPA: membrane potential changed from to −64±2.1 to −71.3±1.4 mV, mean change of −7.3±0.85 mV, n=7; FIG. 4A,B). Even at high concentrations, compound BnOCPA had little effect on membrane potential (300 nM-1 μM; 0.45±0.2 mV, n=18 cells, P=1.3×10⁻⁷) compared to CPA and was significantly less than that caused by CPA. The same compound BnOCPA solution (300 nM) was further tested against synaptic transmission in sister hippocampal slices and confirmed that it was active by abolishing synaptic transmission (FIG. 4C). Thus, in stark contrast to adenosine and CPA, compound BnOCPA activates presynaptic A₁ receptors (FIG. 2) but did not activate postsynaptic receptors, even at concentrations 15 times the IC₅₀ for synaptic transmission (FIG. 4).

If compound BnOCPA binds to postsynaptic A₁ receptors but does not activate them, then it might be expected to act in a manner analogous to a receptor antagonist, preventing activation by other agonists, a property that has been observed for biased agonists at other receptors. To test this theory, compound BnOCPA (300 nM, 10 minutes) was first applied, followed by CPA (300 nM) in the presence of compound BnOCPA. The effects of CPA on membrane potential were significantly (P=2.89×10⁻⁵) reduced by compound BnOCPA (mean hyperpolarisation reduced from −7.3±0.85 mV to 2.7±2 mV; FIG. 4A, B). These results are consistent with compound BnOCPA binding to postsynaptic A₁ receptors, but not activating them.

To test whether BnOPCA blocked K+ channels mediating postsynaptic hyperpolarisation, or in some other way non-specifically interfered with G protein signalling, we applied the GABA_{b} receptor agonist baclofen to CA1 pyramidal cells. BnOCPA had no effect on membrane hyperpolarisation produced by baclofen (FIG. 4D, 4E), confirming that the actions of BnOCPA on postsynaptic membrane potential, likely explained why, in a model of seizure activity with prominent postsynaptic depolarisation that promotes neuronal firing (low Mg²⁺/high K⁺) BnOCPA had little effect. In contrast, equivalent concentrations of CPA completely suppressed neuronal firing (FIG. 4F, G; see also FIG. 3).

### Example 5: BnOCPA Demonstrates Unique Signalling Bias

To investigate the molecular basis for the unprecedented properties of BnOCPA, we generated a recombinant cell system (CHO-K1 cells) expressing the human A₁R (hA₁R). BnOCPA was a potent (IC₅₀ 0.7 nM; Table 1) full agonist at the hA₁R and bound to the receptor with an affinity equal to that of CPA and NECA, and higher than that of adenosine (FIG. 5A, B). Using individual pertussis toxin (PTX)-insensitive variants of individual Gi/o subunits transfected into PTX-treated cells, we observed that adenosine, CPA, NECA and the unbiased agonist HOCPA coupled to a range of Gi/o subunits, and in particular Goa (FIG. 5C to E; Table 1). In stark contrast, BnOCPA had a distinctive and highly selective Gα subunit activation profile and discriminated between the two Go isoforms in being unable to activate Goa (FIG. 5C to E; Table 1). We hypothesised that BnOCPA should therefore reduce the actions of adenosine on the inhibition of cAMP accumulation via Goa. This was indeed the case (FIG. 5F) and had parallels with the antagonising effects of BnOCPA on membrane potential in the CNS (FIG. 4A, B). We excluded the possibility that the actions of BnOCPA and the prototypical A₁R agonists were mediated via β-arrestins using a BRET assay for β-arrestin recruitment. We observed no β-arrestin recruitment at the A₁R using either BnOCPA, CPA or adenosine (data not shown), observations that are consistent with those previously reported for A₁Rs. The lack of β-arrestin recruitment is likely due to the lack of serine and threonine residues in the A₁R cytoplasmic tail, which makes the A₁R intrinsically biased against β-arrestin signalling.

The data from whole-cell patch-clamp recordings showed that BnOCPA did not influence neuronal membrane potential (FIG. 4A, B), while experiments in recombinant hA₁Rs showed that BnOCPA did not activate Goa (FIG. 5C, E). We thus predicted that A₁Rs in the hippocampus, where Go is highly expressed, particularly at extra-synaptic sites, should act via Goa to induce membrane hyperpolarisation. To test this we injected a series of previously validated interfering peptides against Goa and Gob into CA1 pyramidal cells during whole-cell voltage clamp recordings. Introduction of the Goa interfering peptide caused a significant attenuation of the adenosine-induced outward current (FIG. 5G, H), whereas neither the scrambled peptide nor the Gob peptide had any effect on current amplitude (FIG. 5G, H). Thus, membrane potential hyperpolarisation occurs mainly through A₁R activation of Goa. The data from recombinant receptors demonstrating the inability of BnOCPA to activate Goa (FIG. 5C, E) thus explains why BnOCPA did not cause membrane hyperpolarisation, and indeed prevented or reversed the hyperpolarisation induced by CPA or adenosine, respectively (FIG. 4A, B).

### Example 6: The Signalling Bias Displayed by BnOCPA is Reflected in Non-Canonical Binding Modes and a Selective Interaction with Gα Subunits

To understand better the unusual signalling properties of BnOCPA and the highly specific Gα coupling, we carried out dynamic docking simulations to study the basic orthosteric binding mode of BnOCPA in an explicit, fully flexible environment using the active cryo-EM structure of the A₁R (PDB code 6D9H). We compared BnOCPA to the unbiased agonists adenosine and HOCPA, and an antagonist (PSB36) of the A₁R. BnOCPA engaged the receptor with the same fingerprint as adenosine and HOCPA. Further explorations of the BnOCPA docked state using metadynamics revealed interchangeable variations on this fingerprint (namely modes A, B, and C) that could be distinguished by the orientation of the BnOCPA-unique benzyl (Bn) group. Having established the possible BnOCPA binding modes, we examined the respective contribution of the orthosteric agonists, the G protein a subunit α5 (C-terminal) helix (GαCT), and the Gα protein subunit to the empirically-observed G protein selectivity displayed by BnOCPA (Table 1, FIG. 5A-D).

Simulations in the absence of G protein. Firstly, following Dror et al., (PNAS USA, 108, 18684-18689 (2011)) we compared the dynamics of the BnOCPA-bound A₁R with the corresponding dynamics of the receptor bound to either HOCPA, the A₁R antagonist PSB36, or the apo receptor, our hypothesis being that there may be ligand-dependent differences in the way that the intracellular region of the receptor responds in the absence of the G protein. In these simulations the G protein was omitted so that inactivation was possible and so that the results were not G protein-dependent. The BnOCPA binding modes A-C were interchangeable during MD simulations but were associated with distinctly different dynamics, as monitored by changes in a structural hallmark of GPCR activation, the N^{7.49}PXXY^{7.53} motif. Given the high flexibility shown by the BnOCPA benzyl group during the simulations and its lipophilic character, we hypothesized and simulated a further binding (namely Mode D) not explored during MD. This conformation involves a hydrophobic pocket underneath ECL3 which is responsible for the A₁/A_{2A} selectivity. Superimposition of the four BnOCPA binding modes (A-D) revealed the highly motile nature of the benzyl group of BnOCPA under the simulated conditions (data not shown).

Quantification of the N^{7.49}PXXY^{7.53} dynamics revealed that HOCPA, BnOCPA mode A, BnOCPA mode C and the apo receptor show a similar distribution of the RMSD of the conserved N^{7.49}PXXY^{7.53} motif (FIG. 6A). In contrast, the non-canonical BnOCPA binding modes B and D were responsible for a partial transition of the N^{7.49}PXXY^{7.53} backbone from the active conformation to the inactive conformation in a manner analogous with the antagonist PSB36 (FIG. 6B). Overall, the simulations revealed mode D as the most stable BnOCPA pose, while mode B accounted for 3.6 μs out of 21 μs.

Dynamic Docking of GαCT. To simulate the agonist-driven interaction between the A₁R and the G protein, the α5 (C-terminal) helix (GαCT) of the G protein (Gi2, Goa, Gob) was dynamically docked to the HOCPA and BnOCPA-bound active A₁R structure (again lacking G protein). This allowed us to evaluate the effect of different GαCT on the formation of the complex with A₁R to test the hypothesis that, of Goa, Gob and Gi2, only the GαCT of Gob would fully engage with the BnOCPA-bound active A₁R, in line with the empirical observations of G protein selectivity summarized in Table 1. FIG. 7A shows that the GαCT of Gob docked to the A₁R via a metastable state (MS1) relative to the canonical state (CS1), regardless of whether HOCPA or BnOCPA was bound. The CS1 geometry was found to correspond to the canonical arrangement as found in the cryo-EM A₁R:G protein complex, whereas state MS1 resembles the recently reported non-canonical state observed in the neurotensin receptor, believed to be an intermediate on the way to the canonical state. In contrast, FIG. 7B shows that the GαCT of Goa and Gi2 docks to the A₁R to form metastable states MS2 and MS3. MS2 is similar to the β₂-adrenergic receptor:GsCT fusion complex, proposed to be an intermediate on the activation pathway and a structure relevant to G protein specificity. In this case however, it appears to be on an unproductive pathway.

MD simulations on the full G protein. To test the hypothesis that the non-functional BnOCPA:A₁R:Goa complex showed anomalous dynamics, we increased the complexity of the simulations by considering the Gα subunit of the Goa and Gob protein bound to the A₁R:BnOCPA (mode B or D) complex or the Gob protein bound to A₁R:HOCPA (a functional system). The most visible differences between Goa and Gob comprised the formation of transient hydrogen bonds between the α4-β6 and α3-β5 loops of Goa and helix 8 (H8) of the receptor (data not shown). Similar contacts are present in the non-canonical state of the neurotensin receptor:G protein complex. Overall, Goa interacted more with TM3 and ICL2 residues, while TM5 and TM6, along with ICL1, were more engaged by Gob. Interestingly, R291^{7.56} and I292^{8.47}, which are located under the N^{7.49}PXXY^{7.53} motif, showed a different propensity to interact with Goa or Gob. In this scenario, it is plausible that a particular A₁R conformation stabilized by BnOCPA (as suggested by the simulations in the absence of G protein, FIG. 6A-B) may favor different intermediate states during the activation process of Goa and Gob.

### Example 7: Effect of Compound BnOCPA on Heart Rate and Mean Arterial Pressure

One of the major obstacles to the development of clinically useful compounds that target nervous system adenosine A₁ receptors is the strong expression of A₁ receptors in the heart and the subsequent effects on the cardiovascular system when they are activated. Activation of these A₁ receptors is negatively dromotropic (reducing conduction speed in AV node) causing slowing of the sinus rate. There is also depression of atrial (but not ventricular) contractility, and attenuation of the stimulatory effects of catecholamines on the myocardium. The effects of adenosine in the AV node are the consequence of the opening of G_{iβy}-coupled K⁺ channels as well as to a depression of other currents including I_{Ca}.

Given BnOCPA's clear differential effects in a native physiological system, strong Gα bias, unique binding characteristics and selective Gα interaction, it was hypothesised that these properties might circumvent a key obstacle to the development of A1R agonists for therapeutic use—their powerful effects in the cardiovascular system (CVS) where their activation markedly reduces both heart rate and blood pressure. As these CVS effects are likely through Goa, which is expressed at high levels in the heart and plays an important role in regulating cardiac function, the lack of effect of BnOCPA on Goa predicted that BnOCPA would have minimal effects on the CVS.

To test the effects of compound BnOCPA on cardiac physiology, two approaches were taken. Firstly, the effects of adenosine and compound BnOCPA on the rate of contraction of the isolated frog heart were compared. Frogs (Xenopus leavis) were pithed (to remove any central reflexes) and drugs were directly applied to the exposed heart. Application of 30 μM adenosine (about IC₅₀ for mammalian hippocampal synaptic depression) reversibly reduced the heart rate from 42±1.2 BPM to 35.5±1.2 BPM (mean reduction of 6.25±0.6 BPM, about 15% reduction, n=4 frogs). Following recovery from adenosine, 300 nM (about 5 times the IC₅₀ for synaptic depression) of compound BnOCPA was applied and had no significant effect on the heart rate (change 0.6±0.2 BPM), but reduced the effects of subsequent adenosine applications (from a reduction of 6.25 BPM in control conditions to 0.27±0.2 BPM following compound BnOCPA). The prototypical A₁ receptor agonist CPA reduced HR by 6.2±0.5 BPM (n=3) (FIG. 8A). Thus, BnOCP appears not to activate A₁Rs in the heart, but instead behaves like an antagonist in preventing the actions of the endogenous agonist.

To fully investigate the effects of BnOCPA on the mammalian CVS, its effects were measured on heart rate (HR) and mean arterial blood pressure (MAP) in urethane-anaesthetised, spontaneously breathing adult rats (FIG. 8B,C, D). The resting HR of 432±21 BPM (beats per minute) was significantly reduced to 147±12 BPM (about 66%, P=3×10⁻¹¹) by adenosine (1 mg·kg⁻¹). BnOCPA (10 μg·kg⁻¹) had no significant effect on HR (about 6%, 442±20 Vs 416±21 BPM; P=1) but abolished (P=2.7×10⁻⁹) the bradycardic effects of adenosine when co-injected (mean change 51±4 BPM; about 12%; P=0.67). CPA (420 ng·kg⁻¹) significantly decreased HR (from 408±17 to 207±29 BPM; about 50%, P=1.9×10⁻⁸), a decrease that was not significantly different to the effect of adenosine (P=0.12), but was significantly different to the effect of both BnOCPA (P=9.0×10⁻⁹) and adenosine in the presence of BnOCPA (P=6.7×10⁻⁷). The resting MAP of 86±9 mmHg, was significantly reduced (about 47%, 46±4 mmHg; P=1.4×10⁻⁴) by adenosine. BnOCPA had no significant (P=1) effect on MAP (88±11 vs 85±13 mmHg) and also had no significant (P=1) effect on the response to adenosine when co-injected (51±4 mmHg; P=0.01). CPA significantly decreased MAP (from 83±8 to 51±5 mmHg; P=0.016), a decrease that was not significantly different to the effect of adenosine in the absence or presence of BnOCPA (P=0.63 and P=1). *** p<0.001. Volumes of saline equivalent to the drug injections had no effect on either HR or MAP and there was no waning in the effects of adenosine responses with repeated doses (data not shown). Thus, BnOCPA does not appear to act as an agonist at CVS A₁Rs but instead antagonises the bradycardic effects of A₁Rs on the heart.

The effects on both the isolated frog heart and ventilated rat are consistent and are similar to the effects of compound BnOCPA observed for membrane potential hyperpolarisation in hippocampal neurons. Compound BnOCPA has little or no effect on heart rate and MAP, but blocks the effects of agonists which activate A₁ receptors. The lack of effect on the cardiovascular system increases the usefulness of compound BnOCPA as a lead compound for the development of new A₁ receptor ligands for nervous system disorders.

### Example 8: Respiration

Since adverse effects on respiration (dyspnea) limit the use of systemic A1R agonists, we additionally examined the effects of BnOCPA on respiration. In urethane-anaesthetised, spontaneously breathing adult rats, intravenous injection of the selective A1R agonist CPA caused significant respiratory depression. In stark contrast, BnOCPA had no appreciable effect on respiration (FIG. 9A-D).

### Example 9: Pain

The lack of effect of BnOCPA on the CVS and respiration prompted an investigation into a potential application of A₁R agonists that had previously been severely curtailed by adverse cardiorespiratory events: A₁R agonists as analgesics.

FIG. 10 shows the effects of BnOCPA and CPA on synaptic transmission in spinal nociceptive (pain sensing) dorsal root afferents impinging on neurones of the dorsal horn of the spinal cord of both the rat (A, B) and non-human primate (macaque, C, D). BnOCPA (FIG. 10A) strongly suppressed electrically-evoked dorsal root inputs to dorsal horn neurones. For comparison, similar actions of the prototypical A₁R agonist CPA are shown in FIG. 10B. FIG. 10 C, D shows the effects of BnOCPA at corresponding neurones in the non-human primate spinal cord in suppressing dorsal root inputs to the spinal cord. The suppression of this activity would lead to analgesia. From FIG. 5, to do so with CPA would cause profound decreases in blood pressure and heart rate, whereas BnOCPA would have no such effects.

### Example 10: In Vivo Pain Model

To further test BnOCPA's potential as an analgesic, we used a rat model of chronic neuropathic pain (spinal nerve ligation) a feature of which is mechanical allodynia whereby the affected limb is rendered sensitive to previously innocuous tactile stimuli. Both intravenous (FIG. 9E) and intracathecal (FIG. 9F) BnOCPA potently reversed mechanical allodynia in a dose-dependent manner but had no depressant effects on motor function that might be mistaken for true analgesia (data not shown). Thus, BnOCPA exhibits powerful analgesic properties at doses devoid of cardiorespiratory effects and at several orders of magnitude lower than the non-opioid analgesics pregabalin and gabapentin.

### CONCLUSIONS

The actions of the atypical A₁ adenosine receptor agonist, compound BnOCPA, were characterised on synaptic transmission, membrane potential hyperpolarisation, seizure activity in the hippocampus and spinal nociceptive afferents. The actions were compared to well-established ligands (adenosine, CPA and NECA). All agonists inhibited synaptic transmission, increased paired-pulse facilitation and these effects were blocked by the A₁ receptor antagonist 8-CPT. Thus, the actions of all of the agonists were consistent with the activation of presynaptic A1 receptors.

It has been found that compound BnOCPA does not hyperpolarise the membrane potential of pyramidal cells unlike adenosine and CPA. Even at very high concentrations (up to 1 μM, some 15 times the IC₅₀ against synaptic transmission) it had no effect. Compound BnOCPA does bind to postsynaptic A₁ receptors as it reduces the membrane potential hyperpolarisation produced by CPA and reverses the effects of adenosine. Thus, compound BnOCPA can distinguish between pre- and postsynaptic A₁ receptors being a potent agonist at presynaptic receptors but acts in a manner analogous to an antagonist at postsynaptic A₁ receptors.

Compound BnOCPA had little effect on a model of seizure activity, unlike other prototypical adenosine receptor agonists (CPA, NECA and adenosine) which abolished activity. There are at least two processes that would be expected to contribute to the seizure suppression produced by an A₁ receptor agonist: the inhibition of synaptic transmission and the hyperpolarisation of the membrane potential leading to a reduction in action potential firing. In the seizure model used, the activity is driven mainly by action potential firing and thus the weak effects of compound BnOCPA are consistent with its inability to hyperpolarise neuronal membrane potential.

Using both the isolated frog heart and the anaesthetised ventilated rat preparation, the same effects were observed in both preparations: clear depressant effects of adenosine and the prototypical A₁ receptor agonist CPA on heart rate with no significant effects of compound BnOCPA. A marked reduction in the effects of adenosine on heart rate following the application of compound BnOCPA was also observed, which is consistent with the antagonistic effect of compound BnOCPA on membrane hyperpolarisation induced by adenosine and CPA observed in the hippocampus. Thus, BnOCPA does not activate the A₁ receptors on the heart and also reduces the activation of these receptors by other A₁ receptor agonists, including the endogenous agonist, adenosine.

Thus, native A₁Rs can be induced to signal via distinct signalling pathways and have identified a novel chemotype capable of doing so.

It will be appreciated by those skilled in the art that several variations to the aforementioned embodiments are envisaged without departing from the scope of the invention.

It will also be appreciated by those skilled in the art that any number of combinations of the aforementioned features and/or those shown in the appended drawings provide clear advantages over the prior art and are therefore within the scope of the invention described herein.

1-15. (canceled)

16. A compound of Formula (I) for use in the treatment of a nervous system disorder or pain, wherein Formula (I) is: 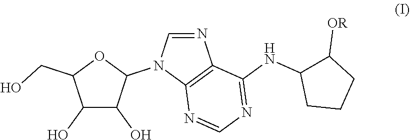
or a pharmaceutically acceptable salt or isomer thereof, wherein:
R is independently hydrogen or R¹R²R³, wherein:
R¹ is independently C₁₋₁₀ alkyl;
R² is independently aryl; and
R³ is independently hydrogen, OH, C(O)NH₂, linear or branched C₁-C₁₀ alkyl, or C₃-C₈ cycloalkyl.

17. The compound for use according to claim 16, wherein:
(i) R¹ is CH₂; and/or
(ii) R² is phenyl; and/or
(iii) R³ is hydrogen, OH, C(O)NH₂, linear or branched C₄-C₁₀ alkyl, or C₃-C₈ cycloalkyl.

18. The compound for use according to claim 16, wherein the compound is selected from the group consisting of: 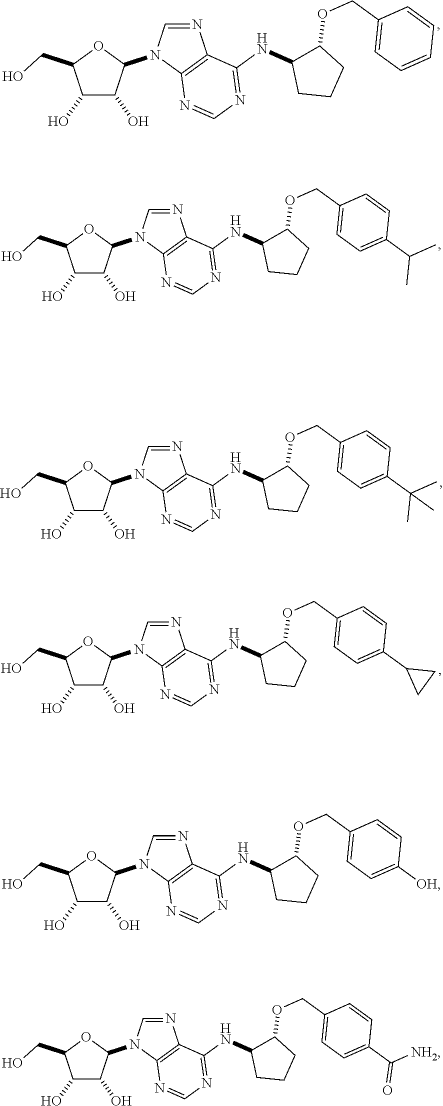
or a pharmaceutically acceptable salt thereof.

19. The compound for use according to claim 16, wherein the nervous system disorder is selected from the group consisting of: epilepsy, ischemia, stroke, traumatic brain injury (TBI), hypoxia, Parkinson's disease, Alzheimer's disease, Huntington's disease, multiple sclerosis, cerebral palsy, encephalitis, meningitis, adrenoleukodystrophy, amyotrophic lateral sclerosis, phenylketonuria, spinal cord injury, dementia, schizophrenia and sleep disorders including insomnia.

20. The compound for use according to claim 16, wherein the compound does not activate the Gα protein subunit Gob.

21. The compound for use according to claim 16, wherein the compound acts as an antagonist of post-synaptic A₁Rs.

22. The compound for use according to claim 16, wherein the compound is not capable of inducing membrane hyperpolarisation.

23. The compound for use according to claim 16, wherein said use does not cause at least one of bradycardia, hypotension and dyspnea.

24. The compound for use according to claim 16, wherein said use comprises administering the compound to a subject who is suffering from, at risk of or in need of treatment for a cardiovascular or respiratory disease.

25. The compound for use according to claim 16, wherein the pain is selected from the group consisting of: neuropathic, nociceptive, peripheral acute and chronic, somatic, visceral, neuroma, diabetic neuropathy, surgical pain, chemotherapy-induced pain, bone pain, inflammatory, phantom limb, myalgia, and multiple sclerosis-related pain; optionally wherein the bone pain is fracture pain or cancer pain.

26. A compound of Formula (I), 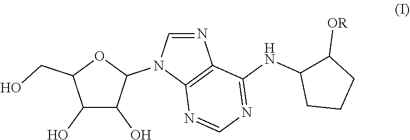
or a pharmaceutically acceptable salt or isomer thereof and wherein R is R¹R²R³, and wherein:
i. R¹ is CH₂;
ii. R² is aryl; and
iii. R³ is independently hydrogen, OH, C(O)NH₂, linear or branched C₁-C₁₀ alkyl, or C₃-C₈ cycloalkyl.

27. A compound according to claim 26, wherein the compound is not:

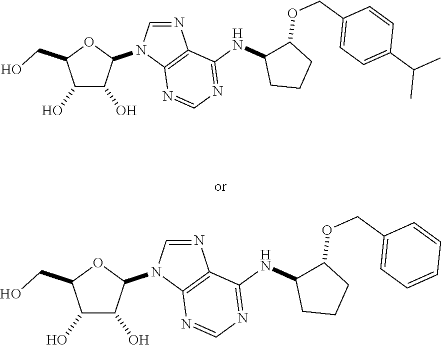

28. A compound according to claim 26, wherein the compound is selected from the group consisting of: 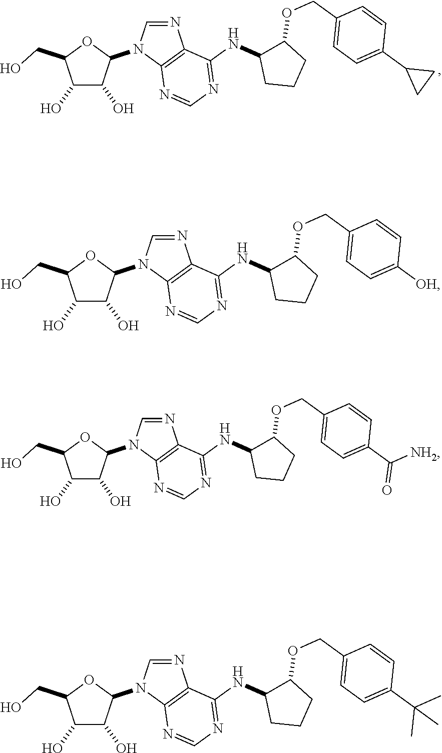
or a pharmaceutically acceptable salt or isomer thereof.

29. A compound according to claim 26, for use as a medicament.

30. A pharmaceutical composition comprising a compound of Formula (I), according to claim 16, and a pharmaceutically or therapeutically acceptable excipient or carrier.

31. A pharmaceutical composition comprising a compound of Formula (I), according to claim 26, and a pharmaceutically or therapeutically acceptable excipient or carrier.

32. A method of treating a disease or condition, comprising the step of administering a therapeutically effective amount of the compound I of claim 16 to a patient.

33. A method according to claim 32, wherein the disease or condition is selected from nervous system disorders, cardiovascular disease, respiratory disease and pain.
